(19) United States
(12) Patent Application Publication
(10) Pub. No.: US 2025/0196187 A1
(43) Pub. Date: Jun. 19, 2025

(54) RESIN COMPOSITION

(71) Applicant: CANON MEDICAL SYSTEMS CORPORATION, Tochigi (JP)

(72) Inventors: Masahide IWAKATA, Kawasaki (JP); Shuhei IKEDA, Yokohama (JP); Kento TSUDA, Yokohama (JP)

(73) Assignee: CANON MEDICAL SYSTEMS CORPORATION, Tochigi (JP)

(21) Appl. No.: 18/988,386

(22) Filed: Dec. 19, 2024

(30) Foreign Application Priority Data
Dec. 19, 2023 (JP) 2023-214214
Jan. 18, 2024 (JP) 2024-006408
Dec. 18, 2024 (JP) 2024-223602

(51) Int. Cl. B06B 1/06; A61B 8/00; C08K 3/08; C08K 3/14; C08K 3/22; C08L 63/00

(52) U.S. Cl. CPC B06B 1/067; A61B 8/4281; A61B 8/4444; C08K 3/08; C08K 3/14; C08K 3/22; C08L 63/00; C08K 2003/2227; C08K 2201/003

(57) ABSTRACT

A resin composition according to an embodiment is a resin composition being a precursor of an acoustic matching layer for an ultrasound transducer element included in an ultrasound transducer element unit having an array transducer element including a piezoelectric body and an electrode. The resin composition includes resin and inorganic material particles, while satisfying 2.3≤Z/√ρ≤3.4 where Z denotes an acoustic impedance of the acoustic matching layer, whereas ρ denotes the density of the inorganic material particles.

### CROSS-REFERENCE TO RELATED APPLICATIONS

This application is based upon and claims the benefit of priority from Japanese Patent Application No. 2023-214214, filed on Dec. 19, 2023, Japanese Patent Application No. 2024-006408, filed on Jan. 18, 2024 and Japanese Patent Application No. 2024-223602, filed on Dec. 18, 2024; the entire contents of which are incorporated herein by reference.

### FIELD

Embodiments described herein relate generally to a resin composition.

### BACKGROUND

In ultrasound diagnosis apparatuses, there is a demand that piezoelectric elements and acoustic matching layers be structured as thin films. Further, there is also a demand that the element size of array transducer elements be miniaturized, as frequencies of ultrasound waves to be transmitted/received are becoming higher.

In relation to the above, acoustic matching layers have conventionally been produced by laminating glass sheets, carbon sheets, or the like with the use of an adhesive agent. However, as acoustic matching base materials for acoustic matching layers are becoming thinner, handling of the base materials and the lamination using an adhesive agent are becoming more difficult. In addition, the level of technical difficulty in processing the base materials for acoustic matching layers is also becoming higher.

To cope with the above, possible methods include a method for laminating acoustic matching layers by which, as disclosed in Japanese Patent No. 5415086, an acoustic impedance is adjusted by mixing resin with a filler using high-density ceramics or metal obtained by mixing nanometer-sized particles with micrometer-sized particles, so as to coat the acoustic matching layers with the mixture.

This method, however, is a method limited to situations where the density of the ceramics particles is high. In addition, because the particles having the mutually-different sizes are mixed together according to this method, there is a problem related to maintaining in-plane uniformity of impedances among the thin film acoustic matching layers.

### BRIEF DESCRIPTION OF THE DRAWINGS

FIG. 1 is a diagram illustrating an exemplary configuration of an ultrasound diagnosis apparatus 100 according to an embodiment;

FIG. 2 is a drawing illustrating an exemplary configuration of ultrasound transducer elements according to the embodiment;

FIG. 3 is a chart for explaining a background related to the embodiment;

FIG. 4 is a drawing for explaining a background related to the embodiment;

FIG. 5 is a table for explaining experiment results related to acoustic matching layer compositions according to the embodiment;

FIG. 6 is a table for explaining a method for producing a resin composition according to the embodiment;

FIG. 7 is a drawing illustrating an exemplary configuration of ultrasound transducer elements according to first and second embodiments;

FIG. 8 is a drawing for explaining a background related to an embodiment;

FIG. 9 is a table for explaining a method for producing a resin composition according to the embodiment;

FIG. 10 is a table for explaining experiment results related to acoustic matching layer compositions according to the embodiment;

FIG. 11 is another table for explaining experiment results related to acoustic matching layer compositions according to the embodiment; and

FIG. 12 is a drawing illustrating an exemplary configuration of an ultrasound transducer elements according to a third embodiment.

### DETAILED DESCRIPTION

A resin composition according to an embodiment is a resin composition being a precursor of an acoustic matching layer for an ultrasound transducer element included in an ultrasound transducer element unit having an array transducer element including a piezoelectric body and an electrode. The resin composition includes resin and inorganic material particles, while satisfying 2.3≤Z/√ρ≤3.4 where Z denotes an acoustic impedance of the acoustic matching layer, whereas p denotes the density of the inorganic material particles.

Exemplary embodiments of a resin composition will be explained in detail below, with reference to the accompanying drawings.

### First Embodiment

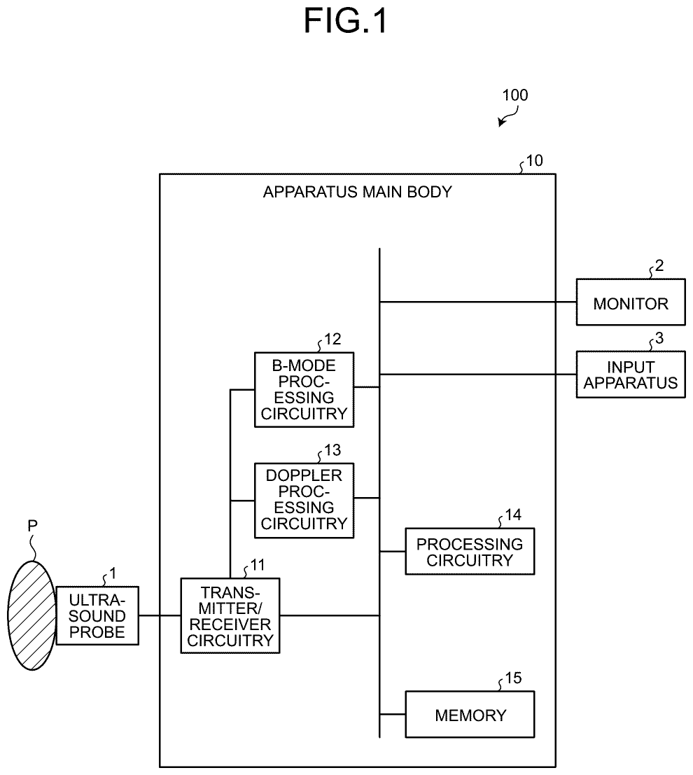

To begin with, with reference to FIG. 1, the following will describe an exemplary configuration of an ultrasound diagnosis apparatus having incorporated therein ultrasound transducer elements produced by using a resin composition according to an embodiment.

As illustrated in FIG. 1, an ultrasound diagnosis apparatus 100 includes an ultrasound probe 1, a monitor 2, an input apparatus 3, and an apparatus main body 10.

The ultrasound probe 1 includes an ultrasound transducer element unit configured to transmit ultrasound waves and to receive reflected waves.

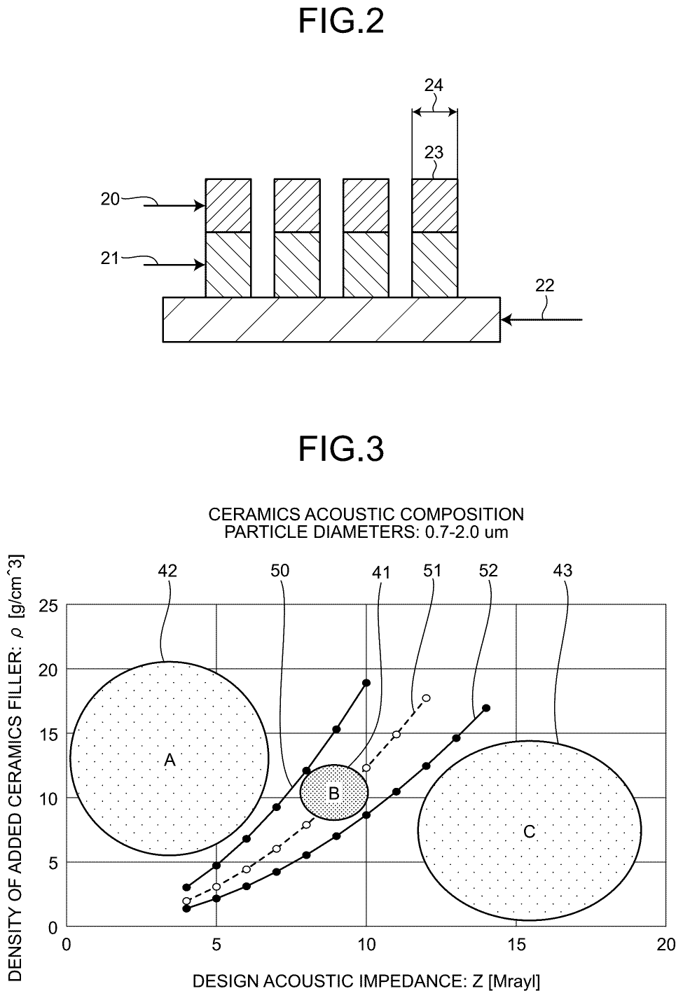

FIG. 2 illustrates a configuration of a part of the ultrasound transducer element unit.

The ultrasound transducer element unit is built in the ultrasound probe 1 and is configured to transmit and receive ultrasound waves to and from a living body with which the probe is in contact. The ultrasound transducer element unit is structured, for example, with a plurality of ultrasound transducer elements 23 that are two-dimensionally arranged; a flexible wiring board (a Flexible Printed Circuit (FPC)) 22 on which the plurality of ultrasound transducer elements 23 are placed; and an acoustic lens. Each of the plurality of ultrasound transducer elements 23 is structured with a piezoelectric element 21, an acoustic matching layer 20, and a back-side matching layer (not illustrated). The element size 24 of each of the ultrasound transducer elements 23 may be, for example, 40 μm.

The piezoelectric element 21 is an element having a piezoelectric characteristic. For example, the piezoelectric element 21 may be a piezoelectric element of PZT (lead zirconate titanate i.e., Pb(Zr,Ti)O₃), PMN-PT (lead magnesium niobate-lead titanate, i.e., Pb(Mg_{1/3}Nb_{2/3})O₃—PbTiO₃), or the like. In an embodiment, the plurality of ultrasound transducer elements 23 are provided while being arranged on a prescribed face of the flexible wiring board 22. A signal electrode is provided on the face (an ultrasound wave emission face) on the side of the piezoelectric element 21 from which an ultrasound wave is emitted. Further, a ground electrode is provided on the face on the opposite side (the back side) of the ultrasound wave emission face side of the piezoelectric element 21.

The piezoelectric element 21 is configured to emit the ultrasound wave from the face on the signal electrode side, as being driven by a drive signal from transmitter/receiver circuitry 11. Further, upon receipt of a reflected wave, the piezoelectric element 21 is configured to convert the received reflected wave into a reflected-wave signal and to output the reflected-wave signal resulting from the conversion, through the signal electrode. The thickness of the piezoelectric element 21 may be 40 μm, for example.

The acoustic matching layer 20 is a layer for acoustically matching the piezoelectric element 21 with the living body, by gradually decreasing an acoustic impedance from the piezoelectric element 21 toward the living body. The acoustic matching layer 20 may be structured with only a single layer or may include a plurality of layers to decrease the acoustic impedance as smoothly as possible toward the living body. As the acoustic matching layer 20, it is possible to use, for example, a resin having an electrically conductive filler mixed therein, in order to achieve electrical conductivity. As the resin, it is possible to use an epoxy resin, for example. The thickness of the acoustic matching layer 20 may be, for example, 30 μm.

Further, the back-side matching layer is structured by using a material having a higher acoustic impedance than that of the piezoelectric elements and is configured, as a resonance layer, to perform the ultrasound transmission/reception in collaboration with the piezoelectric elements.

Further, the flexible wiring board 22 is a Flexible Printed Circuit (FPC) and is configured to electrically connect the signal electrode and the ground electrode to the transmitter/receiver circuitry 11, via wirings provided in the different layers of the ultrasound transducer elements.

Further, a space between the piezoelectric element 21 and the acoustic matching layer 20 may be filled with an adhesive agent, for example. Alternatively, the production may be realized through a lamination coating procedure without using an adhesive agent.

When the ultrasound wave is transmitted from the ultrasound probe 1 to an examined subject (hereinafter, “patient”) P, the transmitted ultrasound wave is repeatedly reflected on a surface of discontinuity of acoustic impedances at a tissue in the body of the patient P and is received as a reflected wave by any of the plurality of ultrasound transducer elements 23 included in the ultrasound probe 1. Each of the reflected waves is converted into a reflected-wave signal being an electric signal, by the piezoelectric element 21 of the ultrasound transducer element 23 that has received the reflected wave. The amplitudes of the reflected-wave signals are dependent on the difference between the acoustic impedances on the surface of discontinuity on which the ultrasound wave is reflected. Further, when a transmitted ultrasound pulse is reflected on the surface of a moving blood flow, a cardiac wall, or the like, the reflected-wave signals are, due to the Doppler effect, subject to a frequency shift, depending on a velocity component of the moving members with respect to the ultrasound wave transmission direction.

The monitor 2 is configured to display a Graphical User Interface (GUI) used by an operator of the ultrasound diagnosis apparatus 100 for inputting various types of setting requests through the input apparatus 3 and to display an ultrasound image or the like generated in the apparatus main body 10.

The input apparatus 3 includes a trackball, a switch, a dial, a touch command screen, and/or the like. The input apparatus 3 is configured to receive the various types of setting requests from the operator of the ultrasound diagnosis apparatus 100 and to transfer the received various types of setting requests to the apparatus main body 10.

The apparatus main body 10 is an apparatus configured to generate an ultrasound image on the basis of the reflected waves received by the ultrasound probe 1, by controlling the ultrasound transmission/reception performed by the ultrasound probe 1. As illustrated in FIG. 1, the apparatus main body 10 includes the transmitter/receiver circuitry 11, B-mode processing circuitry 12, Doppler processing circuitry 13, processing circuitry 14, and a memory 15.

The transmitter/receiver circuitry 11 includes trigger generating circuitry, delay circuitry, pulser circuitry, and the like and is configured to supply the drive signal to the ultrasound probe 1.

The B-mode processing circuitry 12 is configured to generate data (B-mode data) in which signal intensities are expressed with brightness levels, by receiving the reflected-wave data from the transmitter/receiver circuitry 11 and performing a logarithmic amplification, an envelope detection process, and the like.

The Doppler processing circuitry 13 is configured to perform a frequency analysis to obtain velocity information from the reflected-wave data received from the transmitter/receiver circuitry 11, to extract a blood flow, a tissue, and a contrast agent echo component under the Doppler effect, and to generate data (Doppler data) obtained by extracting, with respect to a large number of points, moving member information such as an average velocity, a dispersion value, a power value, and the like.

For example, the processing circuitry 14 is configured by using a controlling processor (a Central Processing Unit (CPU)) that realizes functions of an information processing apparatus (a computer).

By employing an image generating function, the processing circuitry 14 is configured to generate the ultrasound image from the data generated by the B-mode processing circuitry 12 and the Doppler processing circuitry 13. Also, by employing a controlling function, the processing circuitry 14 is configured to control processes performed by the transmitter/receiver circuitry 11, the B-mode processing circuitry 12, the Doppler processing circuitry 13, and the like, on the basis of the various types of setting requests input by the operator via the input apparatus 3 and various types of controlling programs and various types of data read from the memory 15.

The memory 15 is a memory configured to store therein the ultrasound image generated by the processing circuitry 14 while employing an image generating function 14a. Further, the memory 15 is also capable of storing therein any of the data generated by the B-mode processing circuitry 12 and the Doppler processing circuitry 13.

Next, a background of an embodiment will be explained. In the ultrasound diagnosis apparatus 100, for the purpose of improving resolutions of obtained images or for the purpose of obtaining information about a three-dimensional space or the like, there is a tendency that array transducer elements are required to be structured with a larger number of ultrasound transducer elements 23. Meanwhile, the ultrasound probe 1 is required to transmit and receive the ultrasound wave at a higher frequency (a shorter wavelength). Accordingly, there is a tendency that the piezoelectric elements 21, the acoustic matching layers 20, and the like are required to be structured with films that are thinner.

In addition, there is also a demand that the element size 24 of the ultrasound transducer elements 23 be miniaturized, as frequencies of ultrasound waves to be transmitted/received are becoming higher.

In relation to the above, the acoustic matching layers 20 have conventionally been produced by laminating glass sheets, carbon sheets, or the like with the use of an adhesive agent. However, as acoustic matching base materials for the acoustic matching layers 20 are becoming thinner, handling of the base materials and the lamination using an adhesive agent are becoming more difficult. In addition, the level of technical difficulty in processing the base materials for the acoustic matching layers 20 is also becoming higher.

To cope with the above, a process for laminating the acoustic matching layers 20 has been proposed by which, as disclosed in Patent Document 1, an acoustic impedance Z is adjusted by mixing resin with a filler using high-density ceramics or metal obtained by mixing nanometer-sized particles with micrometer-sized particles, so as to coat the acoustic matching layers 20 with the mixture.

The method of Patent Document 1, however, is a method limited to situations where a density p of the ceramics particles is high. In addition, because the particles having the mutually-different sizes are mixed together according to this method, there is a problem related to in-plane uniformity of impedances among the thin film acoustic matching layers.

The resin composition according to an embodiment is based on the background described above. The resin composition according to the embodiment is a resin composition being a precursor of the acoustic matching layers 20 for the ultrasound transducer elements 23 included in the ultrasound transducer element unit having the array transducer elements including a piezoelectric body realized with the piezoelectric elements 21 and the electrodes. The resin composition includes resin and inorganic material particles, while satisfying 2.3≤Z/√ρ≤3.4, where Z denotes the acoustic impedance of the acoustic matching layers 20; and p denotes the density of the ceramics particles. In the present example, in the first embodiment, the inorganic material particles are ceramics particles. In other words, in the first embodiment, the resin composition includes the resin and the ceramics particles, while satisfying 2.3≤Z/√ρ≤3.4, where Z denotes the acoustic impedance of the acoustic matching layers 20; and ρ denotes the density of the ceramics particles.

Next, the value of Z/√ρ will be explained. Generally speaking, when Z denotes an acoustic impedance, whereas p denotes the density of a substance, Z/√ρ expresses a value relevant to a bulk modulus. In other words, Z/√ρ is a value roughly expressing unlikeliness of deformation of the substance. However, it should be noted that, in the embodiment, regarding the resin composition including the resin and the ceramics particles, the value of Z/√ρ is evaluated with respect to the density ρ of the ceramics particles to be added, but not the density of the resin composition itself.

Next, a relationship between values of Z/√ρ and characteristics of the resin composition will be explained, with reference to FIG. 3. FIG. 3 is a graph for explaining the characteristics of the resin composition, while the horizontal axis expresses design values of the acoustic impedance Z, whereas the vertical axis expresses the density ρ of the ceramics particles to be added to the resin.

In the present example, in FIG. 3, curves 50, 51 and 52 are curves satisfying Z/√ρ=2.3, 2.85, and 3.4. The region between the curve 50 and the curve 52, e.g., a region 41 displayed as region B corresponds to an optimal composition of the resin composition. In contrast, a low viscosity region 42 positioned on the left side of the curve 50 and displayed as region A; and a high viscosity region 43 positioned on the right side of the curve 52 and displayed as region C correspond to regions that are not appropriate for the resin composition.

To explain further in detail, the low viscosity region 42 is a region in which the density of the ceramics particles to be added will be high so that sedimentation of the ceramics particles having the high density will occur, which is not desirable. In addition, the low viscosity region 42 is a region in which, because the density of the ceramics particles is high, much reflection scattering and a strong attenuation will occur. In other words, because of the sedimentation of the ceramics particles and the attenuation from the reflection scattering, the low viscosity region 42 is a region that is not appropriate for the resin composition.

On the contrary, in the high viscosity region 43, because a paste having a high viscosity and high thixotropy is formed, mixing, degassing, and spreading/coating processes will technically be difficult. In an example, in the high viscosity region 43, in a uniformly thin film (<100 μm) forming process performed in a coating procedure using slit coating, an applicator, or the like, there will be production-related problems because the level of precision of the coating procedure will not be sufficient. In other words, the high viscosity region 43 is a region that is not appropriate for the resin composition due to the production-related problems.

Consequently, to summarize a first advantage of the resin composition according to the embodiment, satisfying 2.3≤Z/√ρ≤3.4 makes a low attenuation and the coating procedure possible and yields an appropriate resin composition.

In other words, the resin composition according to the embodiment realizes a low attenuation, while realizing an appropriate design range for the acoustic impedance Z, 3.0 MRayl to 15 MRayl. In addition, the resin composition according to the embodiment has a viscosity that makes the coating procedure possible, when a sheer viscosity corresponding to a coating shear rate used by slit coating or an applicator is in the range of 1<Cp<500 (Pas), for example.

Next, a second advantage of the resin composition according to the embodiment will be explained. As for the resin composition according to the embodiment, the resin composition is produced by using the ceramics particles having unimodal particle diameters, instead of mixing particles having mutually-different sizes.

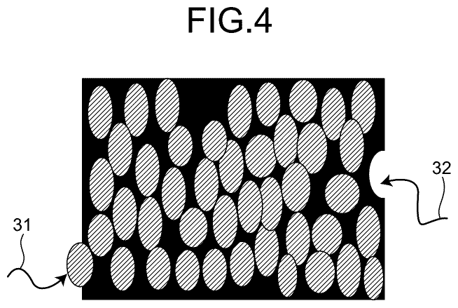

This feature will briefly be explained. As for the particle diameters of the ceramics particles to be added to the resin, the particle diameters are required to be small for the purpose of guaranteeing in-plane uniformity of the impedances among the thin film acoustic matching layers. In this situation, for example, as described in Patent Document 1 or Patent Document 2 (Japanese Patent Application Laid-open No. 2003-169397), when particles having bimodal (the larger and the smaller) particle diameters are to be mixed together, although adjusting the viscosity would be easy, it would be difficult to avoid mixing in a filler having large particle diameters. As a result, in a section where the larger particle diameter filler is present in a larger volume, the acoustic impedance Z would vary in the thickness direction. In addition, as illustrated in FIG. 4, when the large particle diameter filler dispersed in the acoustic matching layers 20 in a dicing step at the time of structuring the ultrasound transducer elements 23 either falls off 32 or remains 31, these phenomena could be a cause of variance in the acoustic impedance Z among the ultrasound transducer elements 23. In particular, for the ultrasound transducer elements 23 designed so that the transmitter ultrasound waves have a high frequency, because the element size 24 is miniaturized, the inter-element variance among the ultrasound transducer elements 23 would be significant. Consequently, it is desirable to configure the particle diameter distribution of an average particle diameter of the ceramics particles according to the embodiment to be a unimodal distribution. When the resin composition is produced by adding ceramics particles having unimodal particle diameters, the in-plane uniformity of the impedance Z of the acoustic matching layers 20 is enhanced, and there will be little variance in the acoustic impedances Z among the array transducer elements.

The resin composition according to the embodiment is a material containing the resin such as an epoxy resin and the ceramics particles while the ceramics particles are uniformly dispersed and being in a form free from micro air bubbles and the like that could be a cause of the attenuation. It is possible to realize the uniform dispersion state by implementing the agitation method described below.

### [About the Ceramics Particles]

As the ceramics particles to be added for a resin composition according to the embodiment, it is possible to widely use ceramics particles that are generally used as a small particle diameter filler. In an example, the ceramics particles contain a substance including at least one selected from the group consisting of Mg, Ca, Ba, B, Al, Y, Hf, Ce, Ti, W, and Si and at least one selected from the group consisting of O, C, N, and S. Typically, the average particle diameter (an average primary particle diameter) of the ceramics particles corresponds to small particle diameters that fall in the range of 0.3 μm to 2.0 μm, inclusive, from the viewpoint of the abovementioned relationship with the viscosity of the acoustic matching layer composition and to prevent the variance in the acoustic impedance among the array transducer elements. In an example, the ceramics particles may include, in an amount of 5% by volume or less, particles of which the particle diameters are in the range of 2.0 μm to 0.005 μm, inclusive. The ceramics particle materials and the content amounts thereof may be adjusted as appropriate so as to satisfy the range of 2.3≤Z/√ρ≤3.4 regarding the design acoustic impedance Z.

### [About the Resin]

Examples of the resin structuring the resin composition according to the embodiment include epoxy resins. More specifically, the examples include commonly-used epoxy resins such as, for instance, aliphatic cyclic (alicyclic) epoxy resins, bisphenol A epoxy resins, bisphenol F epoxy resins, and phenol novolac epoxy resins. Because the filler-dispersed composition is to be used in the coating procedure in the embodiment, it is desirable to use a low viscosity epoxy resin. It is desirable when the viscosity of the epoxy resin is 500 mPas or lower at 24° C.

The epoxy resin used in the embodiment is not particularly limited, and it is possible to widely use one of epoxy resins that are commonly used as a main ingredient of an epoxy-based adhesive agent. Specific examples include, for instance, Celloxide 2021P (registered trademark), which is an aliphatic cyclic (alicyclic) epoxy resin. This resin is characterized by being a low viscosity liquid and having an extremely small chlorine content amount as compared to general-purpose epi-bis type epoxy resins and exhibits an advantage where the product is easily put to use in the coating step.

In this situation, the epoxy resin may be realized with the abovementioned epoxy resin or may contain one or more other epoxy resins in addition to the abovementioned epoxy resin within a range that does not lose the advantageous effects of the present embodiment. It is acceptable to use one type of epoxy resin alone or two or more types of epoxy resins in combination.

As a curing agent, it is possible to use any material known as a curing agent for epoxy resins without particular limitations. In an example, a thermosetting resin may be used as a curing agent for the epoxy resin. Typical examples of the curing agent include, for instance, tertiary amines, imidazole, Lewis acids, Bronsted bases, acid anhydrides, aliphatic amines, aromatic amines, dicyandiamide, dihydrazide compounds, and phenol resins. Among the curing agents capable of keeping the viscosity of the main ingredient low, while avoiding impacting the viscosity of the epoxy resin, catalyst type tertiary amines, imidazole, Lewis acids, and Bronsted bases can easily be put to use. Specific examples thereof that are useful include borate-based cationic polymerization agents, the SI series produced by SAN-AID (registered trademark), and the like.

### [About the Mixing Ratio of the Resin and the Ceramics Particle]

It is desirable to configure the epoxy content amount in the layer material in the embodiment to be in the range of 60% by mass to 80% by mass. Further, it is desirable to configure the ceramics content amount in the layer material in the embodiment to be in the range of 20% by mass to 40% by mass. In other words, typically, the content amount of the ceramics particles in the resin composition is in the range of 20% by volume to 40% by volume, inclusive.

[About the Step of Mixing the Resin with the Ceramics Particles]

Attention should be paid to the following three points regarding the step of mixing the resin with the ceramics particles: Firstly, it needs to be possible to mix the components uniformly; secondly, it needs to be possible to properly eliminate unwanted air bubbles that have entered; and thirdly, the mixing process needs to be performed under a low-temperature condition where it is possible to inhibit a heated curing reaction from starting. As for a mixing method, although it is desirable to perform a kneading process by using a planetary centrifugal mixer equipped with a vacuum bubble elimination mechanism, for example, on such occasion, heat may be generated from friction between the filler particles. An example of a flow in the mixing step will be explained in detail in the embodiments described below.

### [About the Coating Step and the Curing]

Next, the coating step of the acoustic matching layer composition according to the embodiment will be explained. The acoustic matching layer composition refined by implementing the method described above can be used in coating procedures. Specific examples of coating methods include slit coating and coating procedures using an applicator. In an example of a coating procedure using an applicator, it is possible to control the matching layer film thickness with a gap of the applicator or a coating speed.

Additionally, possible structures of the matching layer are not limited to a single-layer structure. For example, after a coating procedure for the first layer is finished, the gap of the applicator may be changed so as to coat the first coated matching layer with the second matching layer, and to further coat the second coated matching layer with the third matching layer. With these processes, it is possible to perform a lamination coating procedure by using the acoustic matching layer composition having multiple acoustic impedances, without using an adhesive agent. In addition, by using a clean oven to apply heat and to cure the matching layer resulting from the coating procedure, it is possible to obtain a sheet-like single-layer or multi-layer acoustic matching sheet.

### [About the Curing Process]

Curing shrinkage occurring at the time of curing the resin matching layer and a linear expansion occurring in a heating step could be a cause of warping of the resin matching layer. For this reason, it is recommended to use, as the curable resin, a room-temperature curable resin or an Ultraviolet (UV) curable resin. However, when a room-temperature curable resin is used like in the example of Patent Document 1, in the step of mixing the resin with the filler, because contact between the filler particles might increase the temperature due to friction heat, a polymerization reaction might start, and the curing might inadvertently occur in some situations. As another example, when a light curable resin is used like in the example of Patent Document 2, because the filler absorbs/scatters light, unevenness in the curing might occur in the depth direction.

In contrast, the coating procedure described above realizes a high-precision coating process that does not trigger warping of the resin acoustic matching layers, even when a thermosetting resin (cured at 150° C.) is used.

An outline of the resin composition according to the embodiment has thus been explained. Next, the resin composition according to the embodiment will be explained with specific examples. As a first example of the first embodiment, we will explain an example in which aluminum oxide (Al2O3) is added, while keeping in mind producing the ultrasound probe 1 of which the center frequency is in the range of 20 MHz to 30 MHz. As a second embodiment, we will explain an example in which tungsten carbide (WC) is added, while keeping in mind producing the ultrasound probe 1 of which the center frequency is approximately 30 MHz.

### First Example of First Embodiment

In the first example of the first embodiment, we will explain the example in which aluminum oxide (Al2O3) is used as the ceramics particles to be added to the resin, while keeping in mind producing the ultrasound probe 1 of which the center frequency is in the range of 20 MHz to 30 MHz.

### [About the Selection of the Ceramics Particles]

To begin with, a reason why aluminum oxide (Al2O3) was selected as the ceramics particles to be added to the resin, when producing the ultrasound probe 1 of which the center frequency is in the range of 20 MHz to 30 MHz, will be explained.

As factors related to the acoustic matching layers, an acoustic impedance design value was set to Z=5.6 (MRayl); a film thickness design value of the acoustic matching layers 20 was set to 30 μm; and a design value of the element size 24 was set to 40 μm.

Further, as a restriction condition, an attenuation coefficient of the acoustic matching layer composition to be caused by reflection scattering of the filler was set to be smaller than 0.6 dB/MHz/mm, under the condition of the center frequency being in the range of 20 MHz to 30 MHz. We explored compositions of the ceramics particles which could be realized by using the design values and the restriction condition presented above.

In this situation, on the basis of the relational expression 2.3≤Z/√ρ≤3.4 between the acoustic impedance Z and the density ρ of the ceramics particles serving as the filler to be added, a composition was calculated while using the intermediate value Z/√ρ=2.85 of the relational expression. When the design value of the acoustic impedance Z=5.6 (MRayl) was assigned thereto, ρ=(3.99 g/cm\hat{ }3) was obtained. In the first example of the first embodiment, as a small particle filler having a density value close to the above value, aluminum oxide (Al2O3: ρ=(3.8 g/cm\hat{ }3)) was selected, while various conditions were taken into consideration, such as being in a commonly-used group of ceramics materials.

### [About the Particle Diameters of the Ceramics Particles]

Next, to discuss the particle diameters of the ceramics particles serving as the filler to be added, as for an upper limit value of the particle diameters of the particles, variance in acoustic characteristics of the array elements would directly be impacted, when a defect in magnitude of 5% or larger is present with respect to matching layer film thickness or the element size. Thus, it is desirable to configure the upper limit value of the particle diameters of the particles so as not to exceed 5% of the matching layer film thickness and the element size. Accordingly, when it is taken into consideration that the matching layer film thickness design value is 30 μm, while the element size design value is 40 μm, it is desirable to configure the particle diameters of the ceramics particles serving as the filler to be added, to be 1.5 μm or smaller.

As a result of the above examination, in the first example of the first embodiment, we selected a commercially-available material of Al2O3 (ρ=(3.8 g/cm\hat{ }3)) having an average particle diameter of 0.7 μm, as the ceramics particles to be added in the first embodiment.

[About Varying the Design Value of the Acoustic Impedance z]

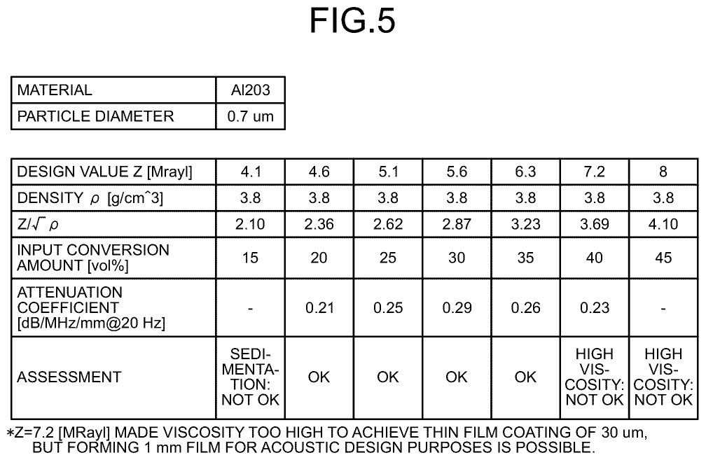

When the density ρ=(3.8 g/cm\hat{ }3) of the filler is assigned to the relational expression 2.3≤Z/√ρ≤3.4 between the acoustic impedance Z and the density ρ of the ceramics particles serving as the filler to be added, 4.48<Z<6.63 (MRayl) is obtained. Accordingly, it is possible to vary the acoustic impedance Z in this range. FIG. 5 presents results of an experiment performed to examine characteristics of the acoustic matching layer compositions produced by varying the value of the acoustic impedance Z in the range of Z=4.1 to 8.

In this situation, at Z=4.1 (MRayl) corresponding to the low viscosity region 42 displayed as region A in FIG. 3, sedimentation of the filler occurred, and the filler was not uniformly dispersed into the outermost layer, so that a thin film layer having only epoxy was formed inadvertently. Thus, the product was not usable as an acoustic matching layer composition. Further, at Z=7.2 to 8.0 (MRayl) corresponding to the high viscosity region 43 displayed as region C in FIG. 3, the viscosity was too high to carry out a high-precision coating procedure (where coating errors <+/−1 μm) with a design film thickness of t=30 μm, by performing a slit coating or implementing an applicator coating method. It should be noted that, because substrates having thicknesses of 1 mm, 2 mm, and 3 mm are used as samples for measuring acoustic impedances, even materials having a high viscosity to a certain extent can be used for the formation thereof and the measuring process. In the region satisfying Z=4.6 to 6.3, the above problem did not occur.

### [About the Resin]

Next, the resin to be mixed with the ceramics particles will be explained. Examples of the resin to be mixed with the ceramics particles include epoxy resins. In the first example of the first embodiment, as an epoxy resin to be mixed with the ceramics particles, Celloxide 2021P (registered trademark) produced by Daicel Corporation (registered trademark) was selected, for example. This material is a liquid epoxy resin having a low viscosity (250 mPas@24° C.) and is suitable for acoustic matching layer compositions for coating purposes.

### [About the Mixing Ratio of the Resin and the Ceramics Particles]

Next, mixing amounts of the resin and the ceramics particles will be explained. By adjusting the mixing ratio of the resin and the ceramics particles as appropriate, it is possible to adjust the value of the acoustic impedance Z. When the density of Al2O3, ρ=(3.8 g/cm\hat{ }3), is assigned to the relational expression 2.3≤Z/√ρ≤3.4 between the acoustic impedance Z and the density ρ of the ceramics particles serving as the filler to be added, 4.48<Z<6.63 (MRayl) is obtained. Accordingly, by varying the mixing amounts of the resin and the ceramics particles, it is possible to adjust the value of the acoustic impedance Z. In an example, in a mixture of “an epoxy+a curing agent+a filler”, when the ceramics particles serving as the filler were added by a volume percentage of 30 vol %, the acoustic impedance Z of the obtained resin composition was Z=5.6 (MRayl).

### [About the Curing Agent]

Next, the curing agent will be explained. Possible examples of the curing agent that can be used include borate-based cationic polymerization agents. In an example, SI-B3A (registered trademark) produced by SAN-AID (registered trademark) was added by 0.15 vol %. In this situation, when being added in a solid form, it would be difficult for a curing agent to be uniformly dispersed in the resin. Thus, it is acceptable, for example, to dilute the curing agent with methyl ethyl ketone (MEK) so that the curing agent is added while satisfying an additive amount of 0.15 vol % and to further eliminate MEK through a vacuum bubble elimination process.

[About the Step of Mixing the Resin with the Ceramics Particles]

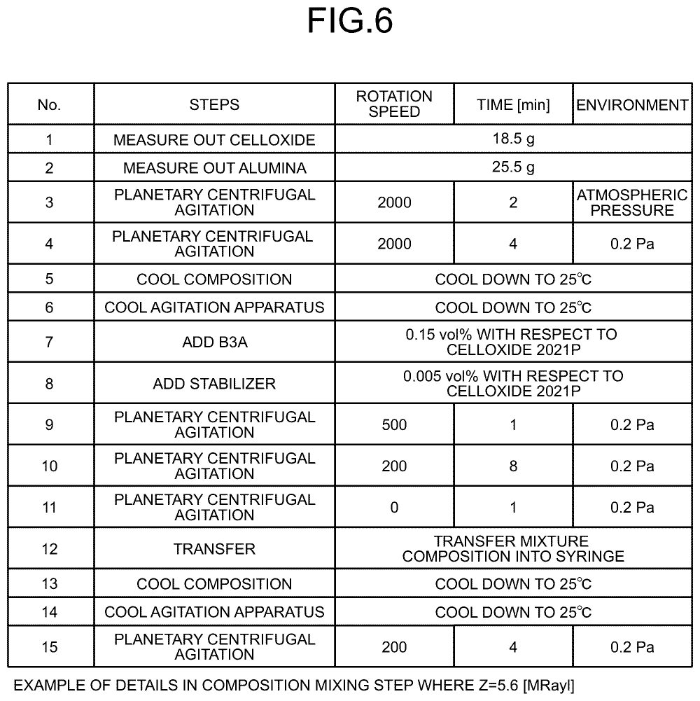

Next, the step of mixing the resin, the ceramics particles serving as the filler, and the curing agent will be explained. FIG. 6 illustrates an example of such a mixing flow. For mixing the resin, the ceramics particles, and the curing agent, possible examples include performing the mixing process by using a planetary centrifugal mixer equipped with a vacuum bubble elimination mechanism.

More specifically, to begin with, as the epoxy resin, 18.6 grams of Celloxide 2021P (registered trademark) produced by Daicel Corporation (registered trademark) was measured out (the first step). Subsequently, 25.5 grams of the Al2O3 filler was measured out (the second step). After that, the Al2O3 filler being the ceramics particles measured out at the second step and Celloxide 2021P (registered trademark) measured out at the first step were uniformly dispersed at 2000 rpm for two minutes (the third step). Subsequently, after the uniform dispersion was confirmed, unwanted micro air bubbles that had entered were eliminated with agitation, again, at 2000 rpm for four minutes in a vacuum of 0.2 Pa (the fourth step). After that, the mixture fluid of the Al2O3 filler and Celloxide 2021P of which the temperature increased due to the high-speed rotation process was cooled down to 25° C. (a room temperature) (the fifth step). Subsequently, the planetary centrifugal mixer of which the temperature increased due to the high-speed rotation process was cooled down to 25° C. (the room temperature) (the sixth step).

After that, the curing agent SI-B3A (0.15 vol %) was dissolved with MEK and added to the composition obtained at the sixth step (the seventh step). Subsequently, a stabilizer (0.005 vol %) was dissolved with ethanol and added to Celloxide 2021P (registered trademark) (the eighth step). However, the eighth step may be omitted. After that, to avoid a temperature increase of the mixture fluid during the rotation, agitation in a vacuum was performed at a low rotation speed (500 rpm) for one minute, so as to eliminate remaining solvents and micro air bubbles (the ninth step). Subsequently, agitation in a vacuum was performed at 200 rpm for eight minutes (the tenth step). After that, we visually checked to see that no bubbles were escaping from the surface of the mixture fluid (the eleventh step), before transferring the mixture composition into a dispense-purpose syringe (the twelfth step). Subsequently, the mixture fluid of the Al2O3 filler and Celloxide 2021P (registered trademark) of which the temperature increased due to the high-speed rotation process was cooled down to 25° C. (the thirteenth step). After that, the planetary centrifugal mixer of which the temperature increased due to the high-speed rotation process was cooled, with a cooling tool, down to 25° C. (the room temperature) (the fourteenth step). Subsequently, air bubbles remaining in the syringe were agitated in a vacuum at 200 rpm for four minutes (the fifteenth step).

The steps described above make it possible to mix, after the curing agent was added, the Al2O3 filler with Celloxide 2021P (registered trademark) at a temperature equal to or lower than 35° C. Under the mixing condition described above, it became possible to control a pot life value (a viscosity change ratio observed two hours after the curing agent was added) of the acoustic matching layer composition satisfying Z=5.6 (MRayl) so as to be smaller than 5%.

In slit coating and applicator coating processes, changes in the viscosity of the composition have a direct impact on the coated film thickness and are therefore not desirable from a viewpoint of film thickness controllability. Through a coating procedure, when we had a matching layer formed with a design matching layer film thickness of 30 μm, we were able to confirm that it was possible to control change amounts in the coated film thickness so as to be smaller than +0.1 μm, with the viscosity increase ratio being smaller than 5% in the presently-used process.

### Second Example of First Embodiment

As a second example of the first embodiment, we will explain an example in which tungsten carbide (WC) is used as the ceramics particles to be added to resin, while keeping in mind producing the ultrasound probe 1 of which the center frequency is 30 MHz]

### [About the Selection of the Ceramics Particles]

To begin with, a reason why tungsten carbide (WC) was selected as the ceramics particles to be added to the resin, when producing the ultrasound probe 1 of which the center frequency is 30 MHz, will be explained.

As factors related to the acoustic matching layers, an acoustic impedance design value was set to Z=11 (MRayl), which is close to that of a glass matching layer; a design value of a matching layer film thickness 21 was set to 40 μm; and a design value of the element size 24 was set to 40 μm.

Further, as a restriction condition, an attenuation coefficient of the acoustic matching layer composition to be caused by reflection scattering of the filler was set to be smaller than 0.6 dB/MHz/mm, under the condition of the center frequency being in the range of 20 MHz to 30 MHz. We explored compositions of the ceramics particles which could be realized by using the design values and the restriction condition presented above.

In this situation, on the basis of the relational expression 2.3≤Z/√ρ≤3.4 between the acoustic impedance Z and the density ρ of the ceramics particles serving as the filler to be added, a composition was calculated while using the intermediate value Z/√ρ=2.85 of the relational expression. When the design value of the acoustic impedance Z=11 (MRayl) was assigned thereto, ρ=(14.9 g/cm\hat{ }3) was obtained. In the second example of the first embodiment, as a small particle filler having a density value close to the above value, WC (ρ=(15.63 g/cm\hat{ }3)) was selected, while various conditions were taken into consideration, such as being in a commonly-used group of ceramics materials.

### [About the Particle Diameters of the Ceramics Particles]

Next, to discuss the particle diameters of the ceramics particles serving as the filler to be added, as for an upper limit value of the particle diameters of the particles, variance in acoustic characteristics of the array elements would directly be impacted, when a defect in magnitude of 5% or larger is present with respect to matching layer film thickness or the element size. Thus, it is desirable to configure the upper limit value of the particle diameters of the particles so as not to exceed 5% of the matching layer film thickness and the element size. Accordingly, when it is taken into consideration that the matching layer film thickness design value is 30 μm, while the element size design value is 40 μm, it is desirable to configure the particle diameters of the ceramics particles serving as the filler to be added, to be 1.5 μm or smaller.

As a result of the above examination, in the second example of the first embodiment, we selected a commercially-available material of WC (ρ=(15.63 g/cm\hat{ }3)) having an average particle diameter of 1.5 μm, as the ceramics particles to be added in the second example of the first embodiment.

### [About the Resin]

Next, the resin to be mixed with the ceramics particles will be explained. Examples of the resin to be mixed with the ceramics particles include epoxy resins. Similarly to the first example of the first embodiment, in the second example of the first embodiment, as an epoxy resin to be mixed with the ceramics particles, Celloxide 2021P (registered trademark) produced by Daicel Corporation (registered trademark) was selected, for example. This material is a liquid epoxy resin having a low viscosity (250 mPas@24° C.) and is suitable for acoustic matching layer compositions for coating purposes.

### [About the Mixing Ratio of the Resin and the Ceramics Particles]

Next, mixing amounts of the resin and the ceramics particles will be explained. By adjusting the mixing ratio of the resin and the ceramics particles as appropriate, it is possible to adjust the value of the acoustic impedance Z. When the density of WC, ρ=(15.63 g/cm\hat{ }3), is assigned to the relational expression 2.3≤Z/√ρ≤3.4 between the acoustic impedance Z and the density ρ of the ceramics particles serving as the filler to be added, 9.1<Z<13.8 (MRayl) is obtained. Accordingly, by varying the mixing amounts of the resin and the ceramics particles, it is possible to adjust the value of the acoustic impedance Z. In an example, in a mixture of “an epoxy+a curing agent+a filler”, when the ceramics particles serving as the filler were added by a volume percentage of 30 vol %, the acoustic impedance Z of the obtained resin composition was Z=11.0 (MRayl).

### [About the Curing Agent]

Next, the curing agent will be explained. Possible examples of the curing agent that can be used include borate-based cationic polymerization agents. In an example, SI-B3A (registered trademark) produced by SAN-AID (registered trademark) was added by 0.15 vol %. In this situation, when being added in a solid form, it would be difficult for a curing agent to be uniformly dispersed in the resin. Thus, it is acceptable, for example, to dilute the curing agent with methyl ethyl ketone (MEK) so that the curing agent is added while satisfying an additive amount of 0.15 vol % and to further eliminate MEK through a vacuum bubble elimination process.

[About the Step of Mixing the Resin with the Ceramics Particles]

Next, the step of mixing the resin, the ceramics particles serving as the filler, and the curing agent will be explained. By performing a mixing step similar to that in the first example of the first embodiment illustrated in FIG. 6, it is possible to mix together the resin, the ceramics particles serving as the filler, and the curing agent. In other words, by performing the mixing process by using a planetary centrifugal mixer equipped with a vacuum bubble elimination mechanism, for example, it is possible to mix together the resin, the ceramics particles serving as the filler, and the curing agent. Although the volume percentage of the ceramics particles to be mixed therein is approximately 30%, similarly to the first example of the first embodiment, because the density of WC serving as the ceramics particles to be mixed therein is larger in the second embodiment, the weight of the material to be mixed therein is larger than that in the example in FIG. 6.

The steps described above make it possible to mix the WC filler with Celloxide 2021P (registered trademark) at a temperature equal to or lower than 35° C. Under the mixing condition described above, it became possible to control a pot life value (a viscosity change ratio observed two hours after the curing agent was added) of the acoustic matching layer composition satisfying Z=11.0 (MRayl) so as to be smaller than 5%.

In slit coating and applicator coating processes, changes in the viscosity of the composition have a direct impact on the coated film thickness and are therefore not desirable from a viewpoint of film thickness controllability. Through a coating procedure, when we had a matching layer formed with a design matching layer film thickness of 30 μm, we were able to confirm that it was possible to control change amounts in the coated film thickness so as to be smaller than +0.1 μm, with the viscosity increase ratio being smaller than 5% in the presently-used process.

According to at least one aspect of the embodiment described above, it is possible to produce the resin composition that has a low attenuation and makes the coating procedure possible.

### Second Embodiment

In the first embodiment, the examples were explained in which the inorganic material particles were ceramics particles. As a second embodiment, an example will be explained in which the inorganic material particles are metal particles.

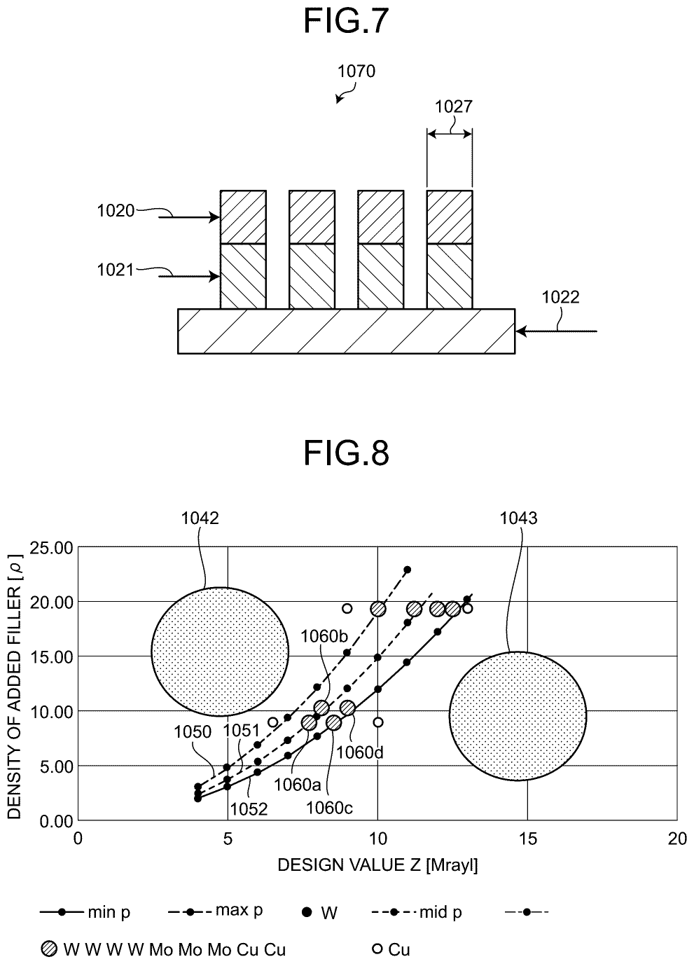

FIG. 7 illustrates an exemplary configuration of a part of an ultrasound transducer element unit 1070 according to a second embodiment.

The ultrasound transducer element unit 1070 is built in the ultrasound probe 1 and is configured to transmit and receive ultrasound waves to and from a living body with which the probe is in contact. The ultrasound transducer element unit 1070 is structured, for example, with a plurality of ultrasound transducer elements that are two-dimensionally arranged; a flexible wiring board (a Flexible Printed Circuit (FPC)) 1022 on which the plurality of ultrasound transducer elements are placed; and an acoustic lens. Each of the plurality of ultrasound transducer elements is structured with a piezoelectric element 1021, an acoustic matching layer 1020, and a back-side matching layer (not illustrated). The element size 1027 of each of the ultrasound transducer elements may be, for example, 40 μm.

The piezoelectric element 1021 is an element having a piezoelectric characteristic. For example, the piezoelectric element 1021 may be a piezoelectric element of PZT (lead zirconate titanate i.e., Pb(Zr,Ti)O3), PMN-PT (lead magnesium niobate-lead titanate, i.e., Pb(Mg_{1/3}Nb_{2/3})O₃—PbTiO₃), or the like. In an embodiment, the plurality of ultrasound transducer elements are provided while being arranged on a prescribed face of the flexible wiring board 1022. A signal electrode is provided on the face (an ultrasound wave emission face) on the side of the piezoelectric element 1021 from which an ultrasound wave is emitted. Further, a ground electrode is provided on the face on the opposite side (the back side) of the ultrasound wave emission face side of the piezoelectric element 1021.

The piezoelectric element 1021 is configured to emit the ultrasound wave from the face on the signal electrode side, as being driven by a drive signal from transmitter/receiver circuitry 1011. Further, upon receipt of a reflected wave, the piezoelectric element 1021 is configured to convert the received reflected wave into a reflected-wave signal and to output the reflected-wave signal resulting from the conversion, through the signal electrode. The thickness of the piezoelectric element 1021 may be 40 μm, for example.

The acoustic matching layer 1020 is a layer for acoustically matching the piezoelectric element 1021 with the living body, by gradually decreasing an acoustic impedance from the piezoelectric element 1021 toward the living body. The acoustic matching layer 1020 may be structured with only a single layer or may include a plurality of layers to decrease the acoustic impedance as smoothly as possible toward the living body. As the acoustic matching layer 1020, it is possible to use, for example, a resin having an electrically conductive filler mixed therein, in order to achieve electrical conductivity. As the resin, it is possible to use an epoxy resin, for example. The thickness of the acoustic matching layer 1020 may be, for example, 30 μm.

Further, the back-side matching layer is structured by using a material having a higher acoustic impedance than that of the piezoelectric elements and is configured, as a resonance layer, to perform the ultrasound transmission/reception in collaboration with the piezoelectric elements.

Further, the flexible wiring board 1022 is a Flexible Printed Circuit (FPC) and is configured to electrically connect the signal electrode and the ground electrode to the transmitter/receiver circuitry 11, via wirings provided in the different layers of the ultrasound transducer elements.

Further, a space between the piezoelectric element 1021 and the acoustic matching layer 1020 may be filled with an adhesive agent, for example. Alternatively, the production may be realized through a lamination coating procedure without using an adhesive agent.

Next, a background of the second embodiment will be explained. In the ultrasound diagnosis apparatus 100, for the purpose of improving resolutions of obtained images or for the purpose of obtaining information about a three-dimensional space or the like, there is a tendency that array transducer elements are required to be structured from a larger number of ultrasound transducer elements. Meanwhile, the ultrasound probe 1 is required to transmit and receive the ultrasound wave at a higher frequency (a shorter wavelength). Accordingly, there is a tendency that the piezoelectric elements 1021, the acoustic matching layers 1020, and the like are required to be structured with films that are thinner.

In addition, there is also a demand that the element size of the ultrasound transducer elements be miniaturized, as frequencies of ultrasound waves to be transmitted/received are becoming higher.

In relation to the above, the acoustic matching layers 1020 have conventionally been produced by laminating glass sheets, carbon sheets, or the like with the use of an adhesive agent. However, as acoustic matching base materials for the acoustic matching layers 1020 are becoming thinner, handling of the base materials and the lamination using an adhesive agent are becoming more difficult. In addition, the level of technical difficulty in processing the base materials for the acoustic matching layers 1020 is also becoming higher.

To cope with the above, a process for laminating the acoustic matching layers 1020 has been proposed by which, as disclosed in Patent Document 1, the acoustic impedance Z is adjusted by mixing resin with high-density ceramics or the like obtained by mixing nanometer-sized particles with micrometer-sized particles, so as to coat the acoustic matching layers 1020 with the mixture.

The method of Patent Document 1, however, is a method limited to situations where the density ρ of the ceramics particles is high. In addition, because the particles having the mutually-different sizes are mixed together according to this method, there is a problem related to in-plane uniformity of impedances among the thin film acoustic matching layers.

A resin composition according to the second embodiment is based on the background described above and is based on a method for adding the metal particles. The resin composition according to the embodiment is a resin composition being a precursor of the acoustic matching layers 1020 for the ultrasound transducer elements 1024 included in the ultrasound transducer element unit having the array transducer elements including a piezoelectric body realized with the piezoelectric elements 1021 and the electrodes. The resin composition includes resin and the metal particles, while satisfying 2.3≤Z/√ρ≤2.9, where Z denotes the acoustic impedance of the acoustic matching layers 1020; and p denotes the density of the metal particles.

Next, the value of Z/√ρ will be explained. Generally speaking, when Z denotes an acoustic impedance, whereas p denotes the density of a substance, Z/√ρ expresses a value relevant to a bulk modulus. In other words, Z/√ρ is a value roughly expressing unlikeliness of deformation of the substance. However, it should be noted that, in the embodiment, regarding the resin composition including the resin and the metal particles, the value of Z/√ρ is evaluated with respect to the density ρ of the metal particles to be added, but not the density of the resin composition itself.

Next, a relationship between values of Z/√ρ and characteristics of the resin composition will be explained, with reference to FIG. 8. FIG. 8 is a graph for explaining the characteristics of the resin composition, while the horizontal axis expresses design values of the acoustic impedance Z, whereas the vertical axis expresses the density ρ of the metal particles to be added to the resin.

In the present example, in FIG. 8, the straight lines 1050 and 1052 are straight lines representing the values Z/√ρ=2.3 and 2.9 serving as the lower limit and the upper limit, respectively, of the inequality expression 2.3≤Z/√ρ≤2.9 presented above. The straight line 1051 is a straight line representing values of p that are medians of the values in the straight lines 1050 and 1052. The region between the straight line 1050 and the straight line 1052, e.g., regions 1060a, 1060b, 1060c, 1060d, etc., correspond to an optimal composition of the resin composition. In contrast, a low viscosity region 1042 positioned on the left side of the straight line 1050 and a high viscosity region 1043 positioned on the right side of the straight line 1052 correspond to regions that are not appropriate for the resin composition.

To explain further in detail, the low viscosity region 1042 is a region in which the density of the metal particles to be added will be high so that sedimentation of the metal particles having the high density will occur, which is not desirable. In addition, in the low viscosity region 1042, a problem arises where a strong attenuation occurs due to reflection scattering from an impact of an average interparticle distance. In other words, because of the sedimentation of the metal particles and the attenuation from the reflection scattering, the low viscosity region 1042 is a region that is not appropriate for the resin composition.

On the contrary, in the high viscosity region 1043, because a paste having a high viscosity and high thixotropy is formed, mixing, degassing, and spreading/coating processes will technically be difficult. In an example, in the high viscosity region 1043, in a uniformly thin film (<100 μm) forming process performed in a coating procedure using slit coating, an applicator, or the like, there will be production-related problems because the level of precision of the coating procedure will not be sufficient. In other words, the high viscosity region 1043 is a region that is not appropriate for the resin composition due to the production-related problems.

Consequently, to summarize a first advantage of the resin composition according to the embodiment, as for the metal particle material according to the embodiment, the relationship between the design acoustic impedance Z and the density ρ of the metal particle material satisfying 2.3≤Z/√ρ≤2.9 makes a low attenuation and the coating procedure possible and yields an appropriate resin composition.

In other words, the resin composition according to the embodiment realizes a low attenuation, while satisfying the appropriate design range for the acoustic impedance Z, 3.0 MRayl to 15 MRayl. In addition, the resin composition according to the embodiment has a viscosity that makes the coating procedure possible, when a sheer viscosity corresponding to a coating shear rate used by slit coating or an applicator is in the range of 1<Cp<500 (Pas), for example.

Next, a second advantage of the resin composition according to the embodiment will be explained. As for the resin composition according to the embodiment, the resin composition is produced by using the metal particles having primarily small particle diameters and unimodal particle diameters, instead of mixing particles having mutually-different sizes.

This feature will briefly be explained. As for the particle diameters of the metal particles to be added to the resin, the particle diameters are required to be small for the purpose of guaranteeing in-plane uniformity of the impedances among the thin film acoustic matching layers. In this situation, for example, as illustrated in Patent Document 1 or Patent Document 2 (Japanese Patent Application Laid-open No. 2003-169397), when particles having bimodal (the larger and the smaller) particle diameters are to be mixed together, although adjusting the viscosity would be easy, it would be difficult to avoid mixing in a filler having large particle diameters. As a result, in a section where the larger particle diameter filler is present in a larger volume, the acoustic impedance Z would vary in the thickness direction. In addition, when the large particle diameter filler dispersed in the acoustic matching layers 1020 in a dicing step at the time of structuring the ultrasound transducer elements either falls off or remains, these phenomena could be a cause of variance in the acoustic impedance Z among the ultrasound transducer elements. In particular, for the ultrasound transducer elements designed so that the transmission ultrasound waves have a high frequency, because the element size is miniaturized, the inter-element variance among the ultrasound transducer elements would be significant. Consequently, it is desirable to configure the particle diameter distribution of an average particle diameter of the metal particles according to the embodiment to be a unimodal distribution with small particle diameters. When the resin composition is produced by adding metal particles having unimodal particle diameters, the in-plane uniformity of the impedance Z of the acoustic matching layers 1020 is enhanced, and there will be little variance in the acoustic impedance Z among the array transducer elements. In other words, it is possible to decrease the variance in the acoustic impedance among the array transducer elements and to also provide the resin composition that has a low attenuation and makes the coating procedure possible.

Further, in the embodiment, while using the single material, it is possible to find a material having an appropriate density and being usable at an appropriate level of powder granularity. In other words, as a result of mixing the resin with the metal particles with an appropriate volume ratio, the coating procedure becomes extremely easy due to the viscosity and due to the formation of the composition with the thixotropic paste. Thus, the production-related problems are solved. Furthermore, it is possible to exercise control to keep low the reflection scattering attenuation that may be caused by the adding of the filler.

In addition, the resin composition according to the embodiment is a material containing the resin such as an epoxy resin and the metal particles while the metal particles are uniformly dispersed and being in a form free from micro air bubbles and the like that could be a cause of the attenuation. It is possible to realize the uniform dispersion state by implementing the agitation method described below.

### [About the Resin]

In relation to the above, examples of the resin structuring the resin composition according to the embodiment include epoxy resins. More specifically, the examples include commonly-used epoxy resins such as, for instance, aliphatic cyclic (alicyclic) epoxy resins, bisphenol A epoxy resins, bisphenol F epoxy resins, and phenol novolac epoxy resins. Because the filler-dispersed composition is to be used in the coating procedure in the embodiment, it is desirable to use a low viscosity epoxy resin. It is desirable when the viscosity of the epoxy resin is 500 mPas or lower at 24° C.

The epoxy resin used in the embodiment is not particularly limited, and it is possible to widely use one of epoxy resins that are commonly used as a main ingredient of an epoxy-based adhesive agent. Specific examples include, for instance, Celloxide 2021P (registered trademark), which is an aliphatic cyclic (alicyclic) epoxy resin. This resin is characterized by being a low viscosity liquid and having an extremely small chlorine content amount as compared to general-purpose epi-bis type epoxy resins and exhibits an advantage where the product is easily put to use in the coating step.

In this situation, the epoxy resin may be realized with the abovementioned epoxy resin or may contain one or more other epoxy resins in addition to the abovementioned epoxy resin within a range that does not lose the advantageous effects of the present embodiment. It is acceptable to use one type of epoxy resin alone or two or more types of epoxy resins in combination.

### [About the Curing Agent]

As the curing agent, it is possible to use any material known as a curing agent for epoxy resins without particular limitations. In an example, a thermosetting resin may be used as the curing agent for the epoxy resin. Typical examples of the curing agent include, for instance, tertiary amines, imidazole, Lewis acids, Bronsted bases, acid anhydrides, aliphatic amines, aromatic amines, dicyandiamide, dihydrazide compounds, and phenol resins. Among the curing agents capable of keeping the viscosity of the main ingredient low, while avoiding impacting the viscosity of the epoxy resin, catalyst type tertiary amines, imidazole, Lewis acids, and Bronsted bases can easily be put to use. Specific examples thereof that are useful include borate-based cationic polymerization agents, the SI series produced by SAN-AID (registered trademark), and the like.

### [About the Metal Particles]

As the metal particles to be added for a resin composition according to the embodiment, it is possible to widely use metal particles that are generally used as a small particle diameter filler. In an example, the metal particles contain a substance including at least one selected from the group consisting of Au, Ag, Pt, Cu, Cr, Zr, Zn, Ta, Ti, Mg, Ni, Ca, Ba, Al, Y, Hf, Ce, Ti, Mo, W, Si, Pd, Ir, Sn, Fe, Pb, Pd, and Nd. Typically, the average particle diameter (an average primary particle diameter) of the metal particles corresponds to small particle diameters that fall in the range of 1.0 μm to 4 μm inclusive, from the viewpoint of the abovementioned relationship with the viscosity of the acoustic matching layer composition and to prevent the variance in the acoustic impedance among the array transducer elements. The metal particle materials and the content amounts thereof may be adjusted as appropriate so as to satisfy the range of 2.3≤Z/√ρ≤2.9 regarding the design acoustic impedance Z. The metal particles may include, in an amount of 5% by volume or less, particles of which the particle diameters are in the range of 2.0 μm to 0.005 μm, inclusive.

### [About the Mixing Ratio of the Resin and the Metal Particles]

It is desirable to configure the epoxy content amount in the layer material in the embodiment to be in the range of 60% by mass to 80% by mass. Further, it is desirable to configure the metal particle content amount in the layer material in the embodiment to be in the range of 20% by mass to 40% by mass. In other words, typically, the content amount of the metal particles in the resin composition is in the range of 20% by volume to 40% by volume, inclusive.

[About the Step of Mixing the Resin with the Metal Particles]

Attention should be paid to the following three points regarding the step of mixing the resin with the metal particles: Firstly, it needs to be possible to mix the components uniformly; secondly, it needs to be possible to properly eliminate unwanted air bubbles that have entered; and thirdly, the mixing process needs to be performed under a low-temperature condition where it is possible to inhibit a heated curing reaction from starting. As for a mixing method, for example, it is acceptable to perform a kneading process by using a planetary centrifugal mixer equipped with a vacuum bubble elimination mechanism.

### [About the Coating Step]

The acoustic matching layer composition refined by implementing the method described above can be used in coating procedures. Specific examples of coating methods include slit coating and coating procedures using an applicator. In an example of a coating procedure using an applicator, it is possible to control the matching layer film thickness with a gap of the applicator or a coating speed.

Additionally, possible structures of the matching layer are not limited to a single-layer structure. For example, after a coating procedure for the first layer is finished, the gap of the applicator may be changed so as to coat the first coated matching layer with the second matching layer, and to further coat the second coated matching layer with the third matching layer. With these processes, it is possible to perform a lamination coating procedure by using the acoustic matching layer composition having multiple acoustic impedances, without using an adhesive agent. In addition, by using a clean oven to apply heat and to cure the matching layer resulting from the coating procedure, it is possible to obtain a sheet-like single-layer or multi-layer acoustic matching sheet.

### [About the Curing Method]

Next, a curing method to be implemented after the resin matching layer coating procedure will be discussed. Curing shrinkage occurring at the time of curing the resin matching layer and a linear expansion occurring in a heating step could be a cause of warping of the resin matching layer. For this reason, it is recommended to use a room-temperature curable resin or a UV curable resin. However, when a room-temperature curable resin is used, for example, in the step of mixing the resin with the filler, because contact between the filler particles might increase the temperature due to friction heat, a polymerization reaction might start, and the curing might inadvertently occur in some situations. As another example, when a light curable resin is used, because the filler absorbs/scatters light, unevenness in the curing might occur in the depth direction.

To cope with the circumstances, we explored curing methods to be implemented after the resin matching layer coating procedure. As a result, we established a high-precision coating process that does not trigger warping of the resin acoustic matching layers, even when a thermosetting resin (cured at 150° C.) is used.

In the following sections, specific embodiments will be explained by using a first example of the second embodiment to a third example of the second embodiment. In the first example of the second embodiment, an example in which copper is selected as the metal particles will be explained. In the second example of the second embodiment, an example will be explained in which tungsten is selected as the metal particles. In the third example of the second embodiment, an example using two acoustic matching layers will be explained.

### First Example of Second Embodiment

In the first example of the second embodiment, we will explain an example using copper (Cu) as the metal particles to be added to the resin, while keeping in mind producing the ultrasound probe 1 of which the center frequency is in the range of 20 MHz to 30 MHz.

### [About the Selection of the Metal Particles]

To begin with, a reason why copper (Cu) was selected as the metal particles to be added to the resin, when producing the ultrasound probe 1 of which the center frequency is in the range of 20 MHz to 30 MHz, will be explained.

As factors related to the acoustic matching layers, an acoustic impedance design value was set to Z=7.6 (MRayl); a film thickness design value of the acoustic matching layers 20 was set to 30 μm; and a design value of an element size 27 was set to 40 μm.

Further, as a restriction condition, an attenuation coefficient of the acoustic matching layer composition to be caused by reflection scattering of the filler was set to be smaller than 0.6 dB/MHz/mm, under the condition of the center frequency being in the range of 20 MHz to 30 MHz. We explored compositions of the metal particles which could be realized by using the design values and the restriction condition presented above.

In this situation, on the basis of the relational expression 2.3≤Z/√ρ≤2.9 between the acoustic impedance Z and the density ρ of the metal particles serving as the filler to be added, a composition was calculated while using the intermediate value Z/√ρ=2.55 of the relational expression. When the design value of the acoustic impedance Z=7.6 (MRayl) was assigned thereto, ρ=(8.883 g·cm\hat{ }3) was obtained. In the first embodiment, as a small particle diameter filler having a density value close to the above value, copper (Cu: ρ=(8.96 g/cm\hat{ }3)) was selected, while various conditions were taken into consideration, such as being in a commonly-used group of materials.

### [About the Particle Diameters of the Metal Particles]

Next, to discuss the particle diameters of the metal particles serving as the filler to be added, as for an upper limit value of the particle diameters of the particles, variance in acoustic characteristics of the array elements would directly be impacted, when a defect in magnitude of 10% or larger is present with respect to matching layer film thickness or the element size. Thus, it is desirable to configure the upper limit value of the particle diameters of the particles so as not to exceed 10% of the matching layer film thickness and the element size. Accordingly, when it is taken into consideration that the matching layer film thickness design value is 30 μm, while the element size design value is 40 μm, it is desirable to configure the particle diameters of the metal particles serving as the filler to be added, to be 3 μm or smaller.

As a result of the above examination, in the first example of the second embodiment, we selected a commercially-available material of Cu (ρ=(8.96 g/cm\hat{ }3)) having an average particle diameter of 2.1 μm, as the metal particles to be added in the first example of the second embodiment.

### [About the Resin]

Next, the resin to be mixed with the metal particles will be explained. Examples of the resin to be mixed with the metal particles include epoxy resins. In the first example of the second embodiment, as an epoxy resin to be mixed with the metal particles, an aliphatic cyclic (alicyclic) epoxy resin, such as Celloxide 2021P (registered trademark) produced by Daicel Corporation (registered trademark) was selected, for example. This material is a liquid epoxy resin having a low viscosity (250 mPas@24° C.) and is suitable for acoustic matching layer compositions for coating purposes.

### [About the Mixing Ratio of the Resin and the Metal Particles]

Next, mixing amounts of the resin and the metal particles will be explained. By adjusting the mixing ratio of the resin and the metal particles as appropriate, it is possible to adjust the value of the acoustic impedance Z. When the density of copper, ρ=(8.96 g/cm\hat{ }3), is assigned to the relational expression 2.3≤Z/√ρ≤2.9 between the acoustic impedance Z and the density ρ of the metal particles serving as the filler to be added, 6.88<Z<8.68 (MRayl) is obtained. Accordingly, by varying the mixing amounts of the resin and the metal particles, it is possible to adjust the value of the acoustic impedance Z. In an example, in a mixture of “an epoxy+a curing agent+a filler”, when the metal particles serving as the filler were added by a volume percentage of 33 volt, the acoustic impedance Z of the obtained resin composition was Z=7.6 (MRayl).

### [About the Curing Agent]

Next, the curing agent will be explained. Possible examples of the curing agent that can be used include borate-based cationic polymerization agents. In an example, SI-B3A (registered trademark) produced by SAN-AID (registered trademark) was added by 0.15 vol %. In this situation, when being added in a solid form, it would be difficult for a curing agent to be uniformly dispersed in the resin. Thus, the curing agent was diluted with acetone, for example, so that the curing agent was added while satisfying an additive amount of 0.15 volt, and acetone was eliminated by using a vacuum bubble eliminating method.

[The Step of Mixing the Resin with the Metal Particles]

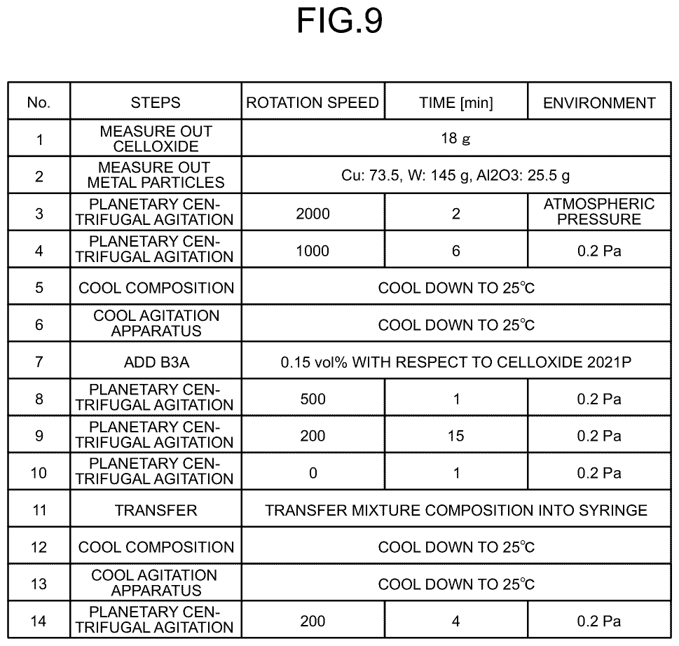

Next, the step of mixing the resin, the metal particles serving as the filler, and the curing agent will be explained. FIG. 9 illustrates an example of such a mixing flow. For mixing the resin, the metal particles, and the curing agent, possible examples include performing the mixing process by using a planetary centrifugal mixer equipped with a vacuum bubble elimination mechanism.

More specifically, to begin with, as the epoxy resin, 18 grams of Celloxide 2021P (registered trademark) produced by Daicel Corporation (registered trademark) was measured out (the first step). Subsequently, 73.5 grams of the copper filler was measured out (the second step). After that, the copper filler being the metal particles measured out at the second step and Celloxide 2021P (registered trademark) measured out at the first step were uniformly dispersed at 2000 rpm for two minutes (the third step). Subsequently, after the uniform dispersion was confirmed, unwanted micro air bubbles that had entered were eliminated with agitation, again, at 1000 rpm for six minutes in a vacuum of 0.2 Pa (the fourth step). After that, the mixture fluid of the copper filler and Celloxide 2021P of which the temperature increased due to the high-speed rotation process was cooled down to 25° C. (a room temperature) (the fifth step). Subsequently, the planetary centrifugal mixer of which the temperature increased due to the high-speed rotation process was cooled down to 25° C. (the room temperature) (the sixth step).

After that, the initiator SI-B3A (0.15 vol %) was dissolved with an acetone solution and added to the composition obtained at the sixth step (the seventh step). Depending on the situations, a stabilizer or a retarder may be used for an adjustment purpose. Subsequently, to avoid a temperature increase of the mixture fluid during the rotation, agitation in a vacuum was performed at a low rotation speed (500 rpm) for one minute, so as to eliminate remaining solvents and micro air bubbles (the eighth step). After that, agitation in a vacuum was performed at 200 rpm for fifteen minutes (the ninth step). Subsequently, we visually checked to see that no bubbles were escaping from the surface of the mixture fluid (the tenth step), before transferring the mixture composition into a dispense-purpose syringe (the eleventh step). After that, the mixture fluid of the copper filler and Celloxide 2021P (registered trademark) of which the temperature increased due to the high-speed rotation process was cooled down to 25° C. (the twelfth step). Subsequently, the planetary centrifugal mixer of which the temperature increased due to the high-speed rotation process was cooled, with a cooling tool, down to 25° C. (the room temperature) (the thirteenth step). After that, air bubbles remaining in the syringe were agitated in a vacuum at 200 rpm for four minutes (the fourteenth step).

The steps described above make it possible, after the SI-B3A serving as the initiator was added, to mix the copper filler with Celloxide 2021P (registered trademark) at a temperature equal to or lower than 35° C. As a result of making it possible to perform the mixing at the low temperature, it was possible to prevent the polymerization reaction from starting and to inhibit viscosity changes (the pot life) in the acoustic matching layer composition.

In slit coating and applicator coating processes, changes in the viscosity of the composition have a direct impact on the coated film thickness and are therefore not desirable from a viewpoint of film thickness controllability. Through a coating procedure, when we had a matching layer formed with a design matching layer film thickness of 40 μm, we confirmed that it was possible to control change amounts in the coated film thickness so as to be smaller than +0.2 μm, with the viscosity increase ratio being smaller than 10% in the presently-used mixing process, one hour after the initiator was added.

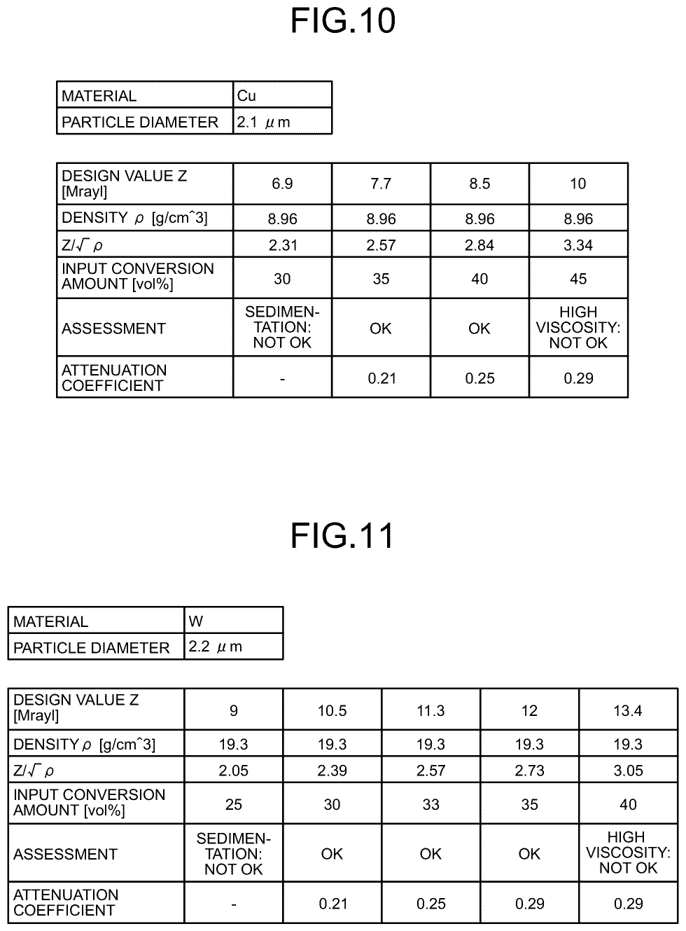

Further, as described above, it is possible to adjust the acoustic matching layer composition in accordance with the design impedance, within the range of the relational expression 2.3≤Z/√ρ≤2.9 between the design acoustic impedance and the filler density values presented above. In other words, when the copper filler satisfying ρ=(8.96 g/cm\hat{ }3) is selected, it is possible to form an acoustic matching layer composition satisfying the range of 6.88<Z<8.68 (MRayl) regarding the acoustic impedance Z. FIG. 10 presents results of an experiment in which the design acoustic impedance was varied with respect to the acoustic matching layer composition using the copper filler.

A list of compositions and evaluation results with respect to the design impedance values will be presented. In a composition region (Z=6.9 (MRayl)) corresponding to the low viscosity region 42 illustrated in FIG. 8, sedimentation of the filler occurred, and the filler was not uniformly dispersed into the outermost layer, so that a thin film layer having only epoxy was formed inadvertently. Thus, we learned that the product was not usable as an acoustic matching layer composition. Further, in a composition region (Z=10.0 (MRayl)) corresponding to the high viscosity region 43 illustrated in FIG. 8, the viscosity was too high to carry out a high-precision coating procedure (where coating errors <+/−1 μm) with a design film thickness of t=40 μm, by performing the slit coating or implementing the applicator coating method described above. It should be noted that, because substrates having thicknesses of 1 mm, 2 mm, and 3 mm are used as samples for measuring acoustic impedances, even materials having a high viscosity to a certain extent can be used for the formation thereof and the measuring process.

### Second Example of Second Embodiment

In a second example of the second embodiment, we will explain an example using tungsten (W) as the metal particles to be added to the resin, while keeping in mind producing the ultrasound probe 1 of which the center frequency is approximately 30 MHz.

### [About the Selection of the Metal Particles]

To begin with, a reason why tungsten (W) was selected as the metal particles to be added to the resin, when producing the ultrasound probe 1 of which the center frequency is approximately 30 MHz will be explained.

As factors related to the acoustic matching layers, an acoustic impedance design value was set to Z=11.2 (MRayl), which is close to that of a glass matching layer; a film thickness design value of the acoustic matching layers 20 was set to 40 μm; and a design value of the element size 24 was set to 40 μm.

Further, as a restriction condition, an attenuation coefficient of the acoustic matching layer composition to be caused by reflection scattering of the filler was set to be smaller than 0.6 dB/MHz/mm, under the condition of the center frequency being in the range of 20 MHz to 30 MHz. We explored compositions of the metal particles which could be realized by using the design values and the restriction condition presented above.

In this situation, on the basis of the relational expression 2.3≤Z/√ρ≤2.9 between the acoustic impedance Z and the density ρ of the metal particles serving as the filler to be added, a composition was calculated while using the intermediate value Z/√ρ=2.55 of the relational expression. When the design value of the acoustic impedance Z=11.2 (MRayl) was assigned thereto, ρ=(19.291 g/cm\hat{ }3) was obtained. In the second example of the second embodiment, as a small particle diameter filler having a density value close to the above value, tungsten (W: ρ=(19.3 g/cm\hat{ }3)) was selected, while various conditions were taken into consideration, such as being in a commonly-used group of materials.

### [About the Particle Diameters of the Metal Particles]

Next, to discuss the particle diameters of the metal particles serving as the filler to be added, as for an upper limit value of the particle diameters of the particles, variance in acoustic characteristics of the array elements would directly be impacted, when a defect in magnitude of 10% or larger is present with respect to matching layer film thickness or the element size. Thus, it is desirable to configure the upper limit value of the particle diameters of the particles so as not to exceed 10% of the matching layer film thickness and the element size. Accordingly, when it is taken into consideration that the matching layer film thickness design value is 40 μm, while the element size design value is 40 μm, it is desirable to configure the particle diameters of the metal particles serving as the filler to be added, to be 4 μm or smaller.

As a result of the above examination, in the second example of the second embodiment, we selected a commercially-available material of W (ρ=(19.3 g/cm\hat{ }3)) having an average particle diameter of 2.2 μm, as the metal particles to be added in the second example of the second embodiment.

### [About the Resin]

Next, the resin to be mixed with the metal particles will be explained. Examples of the resin to be mixed with the metal particles include epoxy resins. In the second example of the second embodiment, as an epoxy resin to be mixed with the metal particles, an aliphatic cyclic (alicyclic) epoxy resin, such as Celloxide 2021P (registered trademark) produced by Daicel Corporation (registered trademark) was selected, for example. This material is a liquid epoxy resin having a low viscosity (250 mPas@24° C.) and is suitable for acoustic matching layer compositions for coating purposes.

### [About the Mixing Ratio of the Resin and the Metal Particles]

Next, mixing amounts of the resin and the metal particles will be explained. By adjusting the mixing ratio of the resin and the metal particles as appropriate, it is possible to adjust the value of the acoustic impedance Z. When the density of tungsten, ρ=(19.3 g/cm\hat{ }3), is assigned to the relational expression 2.3≤Z/√ρ≤2.9 between the acoustic impedance Z and the density ρ of the metal particles serving as the filler to be added, 10.1<Z<12.7 (MRayl) is obtained. Accordingly, by varying the mixing amounts of the resin and the metal particles, it is possible to adjust the value of the acoustic impedance Z. In an example, in a mixture of “an epoxy+a curing agent+a filler”, when the metal particles serving as the filler were added by a volume percentage of 33.2 vol %, the acoustic impedance Z of the obtained resin composition was Z=11.2 (MRayl).

### [About the Curing Agent]

Next, the curing agent will be explained. Possible examples of the curing agent that can be used include borate-based cationic polymerization agents. In an example, SI-B3A (registered trademark) produced by SAN-AID (registered trademark) was added by 0.15 vol %. In this situation, a reason is that, being added in a solid form, it would be difficult for a curing agent to be uniformly dispersed in the resin. Thus, the curing agent was diluted with acetone, for example, so that the curing agent was added while satisfying an additive amount of 0.15 volt, and acetone was eliminated by using a vacuum bubble eliminating method.

[About the Step of Mixing the Resin with the Metal Particles]

Next, the step of mixing the resin, the metal particles serving as the filler, and the curing agent will be explained. To be shared with the first example of the second embodiment, FIG. 9 illustrates an example of a mixing flow in the second example of the second embodiment. For mixing the resin, the metal particles, and the curing agent, possible examples include performing the mixing process by using a planetary centrifugal mixer equipped with a vacuum bubble elimination mechanism.

More specifically, to begin with, as the epoxy resin, 18 grams of Celloxide 2021P (registered trademark) produced by Daicel Corporation (registered trademark) was measured out (the first step). Subsequently, 145 grams of the tungsten filler was measured out (the second step). After that, the tungsten filler being the metal particles measured out at the second step and Celloxide 2021P (registered trademark) measured out at the first step were uniformly dispersed at 2000 rpm for two minutes (the third step). Subsequently, after the uniform dispersion was confirmed, unwanted micro air bubbles that had entered were eliminated with agitation, again, at 1000 rpm for six minutes in a vacuum of 0.2 Pa (the fourth step). After that, the mixture fluid of the tungsten filler and Celloxide 2021P of which the temperature increased due to the high-speed rotation process was cooled down to 25° C. (a room temperature) (the fifth step). Subsequently, the planetary centrifugal mixer of which the temperature increased due to the high-speed rotation process was cooled down to 25° C. (the room temperature) (the sixth step).

After that, the initiator SI-B3A (0.15 vol %) was dissolved with an acetone solution and added to the composition obtained at the sixth step (the seventh step). Depending on the situations, a stabilizer or a retarder may be used for an adjustment purpose. Subsequently, to avoid a temperature increase of the mixture fluid during the rotation, agitation in a vacuum was performed at a low rotation speed (500 rpm) for one minute, so as to eliminate remaining solvents and micro air bubbles (the eighth step). After that, agitation in a vacuum was performed at 200 rpm for fifteen minutes (the ninth step). Subsequently, we visually checked to see that no bubbles were escaping from the surface of the mixture fluid (the tenth step), before transferring the mixture composition into a dispense-purpose syringe (the eleventh step). After that, the mixture fluid of the tungsten filler and Celloxide 2021P (registered trademark) of which the temperature increased due to the high-speed rotation process was cooled down to 25° C. (the twelfth step). Subsequently, the planetary centrifugal mixer of which the temperature increased due to the high-speed rotation process was cooled, with a cooling tool, down to 25° C. (the room temperature) (the thirteenth step). After that, air bubbles remaining in the syringe were agitated in a vacuum at 200 rpm for four minutes (the fourteenth step).

The steps described above make it possible, after the SI-B3A serving as the initiator was added, to mix the tungsten filler with Celloxide 2021P (registered trademark) at a temperature equal to or lower than 35° C. As a result of making it possible to perform the mixing at the low temperature, it was possible to prevent the polymerization reaction from starting and to inhibit viscosity changes (the pot life) in the acoustic matching layer composition.

In slit coating and applicator coating processes, changes in the viscosity of the composition have a direct impact on the coated film thickness and are therefore not desirable from a viewpoint of film thickness controllability. Through a coating procedure, when we had a matching layer formed with a design matching layer film thickness of 40 μm, we confirmed that it was possible to control change amounts in the coated film thickness so as to be smaller than +0.2 μm, with the viscosity increase ratio being smaller than 10% in the presently-used mixing process, one hour after the initiator was added.

Further, as described above, it is possible to adjust the acoustic matching layer composition in accordance with the design impedance, within the range of the relational expression 2.3≤Z/√ρ≤2.9 between the design acoustic impedance and the filler density values presented above. In other words, when the tungsten filler satisfying ρ=(19.3 g/cm\hat{ }3) is selected, it is possible to form an acoustic matching layer composition satisfying 10.1<Z<12.7 (MRayl) regarding the acoustic impedance Z. FIG. 11 presents results of an experiment in which the design acoustic impedance was varied with respect to the acoustic matching layer composition using the tungsten filler.

A list of compositions and evaluation results with respect to the design impedances will be presented. In a composition region (Z=9.0 (MRayl)) corresponding to the low viscosity region 1042 illustrated in FIG. 8, sedimentation of the filler occurred, and the filler was not uniformly dispersed into the outermost layer, so that a thin film layer having only epoxy was formed inadvertently. Thus, we learned that the product was not usable as an acoustic matching layer composition. Further, in a composition region (Z=13.4 (MRayl)) corresponding to the high viscosity region 1043 illustrated in FIG. 8, the viscosity was too high to carry out a high-precision coating procedure (where coating errors <+/−1 μm) with a design film thickness of t=40 μm, by performing the slit coating or implementing the applicator coating method described above. It should be noted that, because substrates having thicknesses of 1 mm, 2 mm, and 3 mm are used as samples for measuring acoustic impedances, even materials having a high viscosity to a certain extent can be used for the formation thereof and the measuring process.

### Third Example of Second Embodiment

In the first example of the second embodiment and the second example of the second embodiment, the examples of producing the single-layer matching layer were explained; however, possible embodiments are not limited to these examples. It is also acceptable to produce an acoustic matching layer including multiple layers. In a third example of the second embodiment, an example will be explained in which the acoustic matching layer includes multiple layers, e.g., being a two-layer matching layer. When the acoustic matching layer includes multiple layers, for example, after a coating procedure for the first layer is finished, the gap of the applicator may be changed so as to coat the first coated matching layer with the second matching layer, and to further coat the second coated matching layer with the third matching layer. With these processes, it is possible to perform a lamination coating procedure on an acoustic matching layer composition having multiple acoustic impedances, without using an adhesive agent.

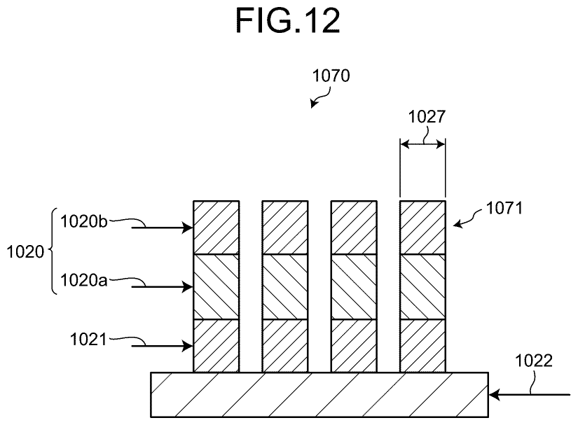

FIG. 12 illustrates a configuration of a part of an ultrasound transducer element unit in an example in which the acoustic matching layer is a two-layer matching layer.

In the third embodiment, the acoustic matching layer 1020 includes a first acoustic matching layer 1020a and a second acoustic matching layer 1020b. In other words, the ultrasound transducer element unit 1070 includes the flexible wiring board 1022, the piezoelectric elements 1021, the first acoustic matching layer 1020a, and the second acoustic matching layer 1020b. In the ultrasound transducer element unit 1070, the element size 1027 of each of the ultrasound transducer elements 1071 may be, for example, 40 μm. The thickness of the piezoelectric element 1021 may be, for example, 40 μm. The thickness of the first acoustic matching layer 1020a may be, for example, 40 μm. The thickness of the second acoustic matching layer 1020b may be, for example, 30 μm.

In the present example, the first acoustic matching layer 1020a is an acoustic matching layer produced by adding metal particles (e.g., tungsten) to a resin. In contrast, the second acoustic matching layer 1020b is an acoustic matching layer produced by adding ceramics particles (e.g., aluminum oxide) to a resin.

However, possible embodiments are not limited to the above example. For example, the order of the first acoustic matching layer 1020a and the second acoustic matching layer 1020b may be opposite. Also, the materials of the first acoustic matching layer 1020a and the second acoustic matching layer 1020b may be other than the above examples.

[About the Selection of the Metal Particles in the First Acoustic Matching Layer 1020a]

In the first acoustic matching layer 1020a, tungsten (W) was used as the metal particles to be added to the resin, on the basis of the examinations presented below.

As factors related to the acoustic matching layers, an acoustic impedance design value was set to Z=11.2 (MRayl); a film thickness design value of the first acoustic matching layer 1020a was set to 40 μm; and a design value of the element size 24 was set to 40 μm.

Further, as a restriction condition, an attenuation coefficient of the acoustic matching layer composition to be caused by reflection scattering of the filler was set to be smaller than 0.6 dB/MHz/mm, under the condition of the center frequency being in the range of 20 MHz to 30 MHz. We explored compositions of the metal particles which could be realized by using the design values and the restriction condition presented above.

In this situation, on the basis of the relational expression 2.3≤Z/√ρ≤2.9 between the acoustic impedance Z and the density ρ of the metal particles serving as the filler to be added, a composition was calculated while using the intermediate value Z/√ρ=2.55 of the relational expression. When the design value of the acoustic impedance Z=11.2 (MRayl) was assigned thereto, ρ=(19.291 g/cm\hat{ }3) was obtained. As a small particle diameter filler having a density value close to the above value, tungsten (W: ρ=(19.3 g/cm\hat{ }3)) was selected, while various conditions were taken into consideration, such as being in a commonly-used group of materials.

### [The First Acoustic Matching Layer 1020a: The Particle Diameters of the Metal Particles]

Next, to discuss the particle diameters of the metal particles serving as the filler to be added, as for an upper limit value of the particle diameters of the particles, variance in acoustic characteristics of the array elements would directly be impacted, when a defect in magnitude of 10% or larger is present with respect to matching layer film thickness or the element size. Thus, it is desirable to configure the upper limit value of the particle diameters of the particles so as not to exceed 10% of the matching layer film thickness and the element size. Accordingly, when it is taken into consideration that the matching layer film thickness design value is 40 μm, while the element size design value is 40 μm, it is desirable to configure the particle diameters of the metal particles serving as the filler to be added, to be 4 μm or smaller.

As a result of the above examination, in the third example of the second embodiment, we selected a commercially-available material of W (ρ=(19.3 g/cm\hat{ }3)) having an average particle diameter of 2.2 μm, as the metal particles to be added in the third example of the second embodiment.

### [The First Acoustic Matching Layer 1020a: About the Resin]

Next, the resin to be mixed with the metal particles will be explained. Examples of the resin to be mixed with the metal particles include epoxy resins. More specifically, as an epoxy resin to be mixed with the metal particles, an aliphatic cyclic (alicyclic) epoxy resin, such as Celloxide 2021P (registered trademark) produced by Daicel Corporation (registered trademark) was selected, for example. This material is a liquid epoxy resin having a low viscosity (250 mPas@24° C.) and is suitable for acoustic matching layer compositions for coating purposes.

### [The First Acoustic Matching Layer 1020a: About the Mixing Ratio of the Resin and Metal Particles]

Next, mixing amounts of the resin and the metal particles will be explained. By adjusting the mixing ratio of the resin and the metal particles as appropriate, it is possible to adjust the value of the acoustic impedance Z. When the density of tungsten, ρ=(19.3 g/cm\hat{ }3), is assigned to the relational expression 2.3≤Z/√ρ≤2.9 between the acoustic impedance Z and the density ρ of the metal particles serving as the filler to be added, 10.1<Z<12.7 (MRayl) is obtained. Accordingly, by varying the mixing amounts of the resin and the metal particles, it is possible to adjust the value of the acoustic impedance Z. In an example, in a mixture of “an epoxy+a curing agent+a filler”, when the metal particles serving as the filler were added by a volume percentage of 33.2 volt, the acoustic impedance Z of the obtained resin composition was Z=11.2 (MRayl).

### [The First Acoustic Matching Layer 1020a: About the Curing Agent]

Next, the curing agent will be explained. Possible examples of the curing agent that can be used include borate-based cationic polymerization agents. In an example, SI-B3A (registered trademark) produced by SAN-AID (registered trademark) was added by 0.15 vol %. In this situation, when being added in a solid form, it would be difficult for a curing agent to be uniformly dispersed in the resin. Thus, the curing agent was diluted with acetone, for example, so that the curing agent was added while satisfying an additive amount of 0.15 vol %, and acetone was eliminated by using a vacuum bubble eliminating method.

[The First Acoustic Matching Layer 1020a: About the Step of Mixing the Resin with the Metal Particles]

Next, the step of mixing the resin, the metal particles serving as the filler, and the curing agent will be explained. As mentioned above, FIG. 9 illustrates an example of such a mixing flow. For mixing the resin, the metal particles, and the curing agent, possible examples include performing the mixing process by using a planetary centrifugal mixer equipped with a vacuum bubble elimination mechanism.

More specifically, to begin with, as the epoxy resin, 18 grams of Celloxide 2021P (registered trademark) produced by Daicel Corporation (registered trademark) was measured out (the first step). Subsequently, 145 grams of the tungsten filler was measured out (the second step). After that, the tungsten filler being the metal particles measured out at the second step and Celloxide 2021P (registered trademark) measured out at the first step were uniformly dispersed at 2000 rpm for two minutes (the third step). Subsequently, after the uniform dispersion was confirmed, unwanted micro air bubbles that had entered were eliminated with agitation, again, at 1000 rpm for six minutes in a vacuum of 0.2 Pa (the fourth step). After that, the mixture fluid of the tungsten filler and Celloxide 2021P of which the temperature increased due to the high-speed rotation process was cooled down to 25° C. (a room temperature) (the fifth step). Subsequently, the planetary centrifugal mixer of which the temperature increased due to the high-speed rotation process was cooled down to 25° C. (the room temperature) (the sixth step).

After that, the initiator SI-B3A (0.15 vol %) was dissolved with an acetone solution and added to the composition obtained at the sixth step (the seventh step). Depending on the situations, a stabilizer or a retarder may be used for an adjustment purpose. Subsequently, to avoid a temperature increase of the mixture fluid during the rotation, agitation in a vacuum was performed at a low rotation speed (500 rpm) for one minute, so as to eliminate remaining solvents and micro air bubbles (the eighth step). After that, agitation in a vacuum was performed at 200 rpm for fifteen minutes (the ninth step). Subsequently, we visually checked to see that no bubbles were escaping from the surface of the mixture fluid (the tenth step), before transferring the mixture composition into a dispense-purpose syringe (the eleventh step). After that, the mixture fluid of the tungsten filler and Celloxide 2021P (registered trademark) of which the temperature increased due to the high-speed rotation process was cooled down to 25° C. (the twelfth step). Subsequently, the planetary centrifugal mixer of which the temperature increased due to the high-speed rotation process was cooled, with a cooling tool, down to 25° C. (the room temperature) (the thirteenth step). After that, air bubbles remaining in the syringe were agitated in a vacuum at 200 rpm for four minutes (the fourteenth step).

The steps described above make it possible, after the SI-B3A serving as the initiator was added, to mix the tungsten filler with Celloxide 2021P (registered trademark) at a temperature equal to or lower than 35° C. As a result of making it possible to perform the mixing at the low temperature, it was possible to prevent the polymerization reaction from starting and to inhibit viscosity changes (the pot life) in the acoustic matching layer composition.

In slit coating and applicator coating processes, changes in the viscosity of the composition have a direct impact on the coated film thickness and are therefore not desirable from a viewpoint of film thickness controllability. Through a coating procedure, when we had a matching layer formed with a design matching layer film thickness of 40 μm, we confirmed that it was possible to control change amounts in the coated film thickness so as to be smaller than +0.2 μm, with the viscosity increase ratio being smaller than 10% in the presently-used mixing process, one hour after the initiator was added.

### [The Second Acoustic Matching Layer 1020b: About the Selection of the Ceramics Particles]

In the second acoustic matching layer 1020b, ceramics particles were used as a substance to be added to the resin, and more specifically, aluminum oxide (Al2O3) was selected.

As factors related to the acoustic matching layers, an acoustic impedance design value was set to Z=5.6 (MRayl); a film thickness design value of the acoustic matching layer 1020b was set to 30 μm; and a design value of the element size 1024 was set to 40 μm.

Further, as a restriction condition, an attenuation coefficient of the acoustic matching layer composition to be caused by reflection scattering of the filler was set to be smaller than 0.6 dB/MHz/mm, under the condition of the center frequency being in the range of 20 MHz to 30 MHz. We explored compositions of the ceramics particles which could be realized by using the design values and the restriction condition presented above.

In this situation, on the basis of the relational expression 2.3≤Z/√ρ≤3.4 between the acoustic impedance Z and the density ρ of the ceramics particles serving as the filler to be added, a composition was calculated while using the intermediate value Z/√ρ=2.85 of the relational expression. When the design value of the acoustic impedance Z=5.6 (MRayl) was assigned thereto, ρ=(3.99 g/cm\hat{ }3) was obtained. In the first embodiment, as a small particle filler having a density value close to the above value, aluminum oxide (Al2O3: ρ=(3.8 g/cm\hat{ }3)) was selected, while various conditions were taken into consideration, such as being in a commonly-used group of ceramics materials.

### [The Second Acoustic Matching Layer 1020b: About the Particle Diameters of the Ceramics Particles]

Next, to discuss the particle diameters of the ceramics particles serving as the filler to be added, as for an upper limit value of the particle diameters of the particles, variance in acoustic characteristics of the array elements would directly be impacted, when a defect in magnitude of 5% or larger is present with respect to matching layer film thickness or the element size. Thus, it is desirable to configure the upper limit value of the particle diameters of the particles so as not to exceed 5% of the matching layer film thickness and the element size. Accordingly, when it is taken into consideration that the matching layer film thickness design value is 30 μm, while the element size design value is 40 μm, it is desirable to configure the particle diameters of the ceramics particles serving as the filler to be added, to be 1.5 μm or smaller.

As a result of the above examination, we selected a commercially-available material of Al2O3 (ρ=(3.8 g/cm\hat{ }3)) having an average particle diameter of 0.7 μm, as the ceramics particles to be added, for the second acoustic matching layer 1020b. In this situation, when the density ρ=(3.8 g/cm\hat{ }3) of the filler is assigned to the relational expression 2.3≤Z/√ρ≤3.4 between the acoustic impedance Z and the density ρ of the ceramics particles serving as the filler to be added, 4.48<Z<6.63 (MRayl) is obtained. Accordingly, it is possible to vary the acoustic impedance Z in this range.

### [The Second Acoustic Matching Layer 1020b: About the Resin]

Next, the resin to be mixed with the ceramics particles will be explained. Examples of the resin to be mixed with the ceramics particles include epoxy resins. As an epoxy resin to be mixed with the ceramics particles, Celloxide 2021P (registered trademark) produced by Daicel Corporation (registered trademark) was selected, for example. This material is a liquid epoxy resin having a low viscosity (250 mPas@24° C.) and is suitable for acoustic matching layer compositions for coating purposes.

### [The Second Acoustic Matching Layer 1020b: About the Mixing Ratio of the Resin and the Ceramics Particles]

Next, mixing amounts of the resin and the ceramics particles will be explained. By adjusting the mixing ratio of the resin and the ceramics particles as appropriate, it is possible to adjust the value of the acoustic impedance Z. When the density of Al2O3, ρ=(3.8 g/cm\hat{ }3), is assigned to the relational expression 2.3≤Z/√ρ≤3.4 between the acoustic impedance Z and the density ρ of the ceramics particles serving as the filler to be added, 4.48<Z<6.63 (MRayl) is obtained. Accordingly, by varying the mixing amounts of the resin and the ceramics particles, it is possible to adjust the value of the acoustic impedance Z. In an example, in a mixture of “an epoxy+a curing agent+a filler”, when the ceramics particles serving as the filler were added by a volume percentage of 30 vol %, the acoustic impedance Z of the obtained resin composition was Z=5.6 (MRayl).

### [The Second Acoustic Matching Layer 1020b: About the Curing Agent]

Next, the curing agent will be explained. Possible examples of the curing agent that can be used include borate-based cationic polymerization agents. In an example, SI-B3A (registered trademark) produced by SAN-AID (registered trademark) was added by 0.15 vol %. In this situation, when being added in a solid form, it would be difficult for a curing agent to be uniformly dispersed in the resin. Thus, it is acceptable, for example, to dilute the curing agent with acetone so that the curing agent is added while satisfying an additive amount of 0.15 vol % and to further eliminate acetone through a vacuum bubble elimination process.

[The Second Acoustic Matching Layer 1020b: About the Step of Mixing the Resin with the Ceramics Particles]

Next, the step of mixing the resin, the ceramics particles serving as the filler, and the curing agent will be explained. As described above, FIG. 9 illustrates an example of such a mixing flow. For mixing the resin, the ceramics particles, and the curing agent, possible examples include performing the mixing process by using a planetary centrifugal mixer equipped with a vacuum bubble elimination mechanism.

More specifically, to begin with, as the epoxy resin, 18.6 grams of Celloxide 2021P (registered trademark) produced by Daicel Corporation (registered trademark) was measured out (the first step). Subsequently, 25.5 grams of the Al2O3 filler was measured out (the second step). After that, the Al2O3filler being the ceramics particles measured out at the second step and Celloxide 2021P (registered trademark) measured out at the first step were uniformly dispersed at 2000 rpm for two minutes (the third step). Subsequently, after the uniform dispersion was confirmed, unwanted micro air bubbles that had entered were eliminated with agitation, again, at 2000 rpm for six minutes in a vacuum of 0.2 Pa (the fourth step). After that, the mixture fluid of the Al2O3 filler and Celloxide 2021P of which the temperature increased due to the high-speed rotation process was cooled down to 25° C. (a room temperature) (the fifth step). Subsequently, the planetary centrifugal mixer of which the temperature increased due to the high-speed rotation process was cooled, with a cooling tool, down to 25° C. (the room temperature) (the sixth step).

After that, the curing agent SI-B3A (0.15 vol %) was dissolved with acetone and added to the composition obtained at the sixth step (the seventh step). Subsequently, to avoid a temperature increase of the mixture fluid during the rotation, agitation in a vacuum was performed at a low rotation speed (500 rpm) for one minute, so as to eliminate remaining solvents and micro air bubbles (the eighth step). After that, agitation in a vacuum was performed at 200 rpm for fifteen minutes (the ninth step). Subsequently, we visually checked to see that no bubbles were escaping from the surface of the mixture fluid (the tenth step), before transferring the mixture composition into a dispense-purpose syringe (the eleventh step). After that, the mixture fluid of the Al2O3 filler and Celloxide 2021P (registered trademark) of which the temperature increased due to the high-speed rotation process was cooled down to 25° C. (the twelfth step). Subsequently, the planetary centrifugal mixer of which the temperature increased due to the high-speed rotation process was cooled, with a cooling tool, down to 25° C. (the room temperature) (the thirteenth step). After that, air bubbles remaining in the syringe were agitated in a vacuum at 200 rpm for four minutes (the fourteenth step).

The steps described above make it possible, after the curing agent was added, to mix the Al2O3 filler with Celloxide 2021P (registered trademark) at a temperature equal to or lower than 35° C. Under the mixing condition described above, it became possible to control a pot life value (a viscosity change ratio observed two hours after the curing agent was added) of the acoustic matching layer composition satisfying Z=5.6 (MRayl) so as to be smaller than 5%.

In slit coating and applicator coating processes, changes in the viscosity of the composition have a direct impact on the coated film thickness and are therefore not desirable from a viewpoint of film thickness controllability. Through a coating procedure, when we had a matching layer formed with a design matching layer film thickness of 30 μm, we were able to confirm that it was possible to control change amounts in the coated film thickness so as to be smaller than +0.1 μm, with the viscosity increase ratio being smaller than 5% in the presently-used process.

### [Producing the Two-Layer Matching Layer]

The methods for producing the first matching layer 1020a and the second matching layer 1020b have each thus been explained. Next, a method for producing a matching layer composition as a whole and the like will be explained.

Examples of methods for producing the matching layer composition include producing a matching layer composition by using an applicator. As an example, it is acceptable to use a method by which a smooth and flat substrate is coated, by using the applicator, with the first matching layer 1020a and the second matching layer composition 1020b, so as to produce a sheet of the two-layer matching layer. In this situation, because the compositions have mutually-different viscosity levels and thixotropic characteristics, the coated film thicknesses may vary depending on the coating speed and the gap of the applicator. As a result of exploring coating conditions for the matching layer compositions, we were able to obtain a sheet of the two-layer matching layer where “the first matching layer/the second matching layer=40 μm/30 μm” is satisfied as illustrated in FIG. 12, by coating the smooth and flat substrate with the first matching layer composition with a gap of 70 μm at a coating speed of 10 mm/sec; continuously coating the first matching layer with the second matching layer composition with a gap of 110 μm at a coating speed of 5 mm/sec; applying heat and curing at 130° C.; and peeling the resulting product from the smooth and flat substrate. The obtained sheet of the two-layer matching layer is adhered to the piezoelectric elements, before producing an array where the element size is 40 μm by using a blade dicer. As a result, it is possible to obtain the ultrasound array elements including the two-layer matching structure illustrated in FIG. 12.

In relation to the embodiments described above, the following notes are provided as certain aspects and selective characteristics of the present disclosure.

### Note 1:

A resin composition provided according to at least one aspect of the present disclosure is a resin composition being a precursor of an acoustic matching layer for an ultrasound transducer element included in an ultrasound transducer element unit having an array transducer element including a piezoelectric body and an electrode.

The resin composition includes resin and ceramics particles, while satisfying
2.3≤Z/√ρ≤3.4 where Z denotes an acoustic impedance of the acoustic matching layer, whereas p denotes the density of the ceramics particles.

### Note 2:

Z may be in the range of 3.0 MRayl to 15 MRayl, inclusive.

### Note 3:

A particle diameter distribution of an average particle diameter of the ceramics particles may be a unimodal distribution.

### Note 4:

The content amount of the ceramics particles in the resin composition may be in the range of 20% by volume to 40% by volume, inclusive.

### Note 5:

An average particle diameter of the ceramics particles may be in the range of 0.3 μm to 2 μm, inclusive.

### Note 6:

The ceramics particles may contain a substance comprising at least one selected from the group consisting of Mg, Ca, Ba, B, Al, Y, Hf, Ce, Ti, W, and Si and at least one selected from the group consisting of O, C, N, and S.

### Note 7:

The ceramics particles may include, in an amount of 5% by volume or less, particles of which the particles diameters are in the range of 2.0 μm to 0.005 μm, inclusive.

### Note 8:

The resin may be epoxy resin.

### Note 9:

Thermosetting resin may be used as a curing agent for the epoxy resin.

### Note 10:

The ceramics particles may be realized with oxide aluminum.

### Note 11:

The ceramics particles may be realized with tungsten carbide.

### Note 12:

A resin composition provided according to another aspect of the present disclosure is a resin composition being a precursor of an acoustic matching layer for an ultrasound transducer element included in an ultrasound transducer element unit having an array transducer element including a piezoelectric body and an electrode.

The resin composition includes resin and metal particles, while satisfying
2.3≤Z/√ρ≤2.9
where Z denotes an acoustic impedance of the acoustic matching layer, whereas p denotes the density of the metal particles.

### Note 13:

Z may be in the range of 3.0 MRayl to 15 MRayl, inclusive.

### Note 14:

A particle diameter distribution of an average particle diameter of the metal particles may be a unimodal distribution.

### Note 15:

The content amount of the metal particles in the resin composition may be in the range of 20% by volume to 40% by volume, inclusive.

### Note 16:

An average particle diameter of the metal particles may be in the range of 1.0 μm to 4 μm, inclusive.

### Note 17:

The metal particles may contain a substance comprising at least one selected from the group consisting of Au, Ag, Pt, Cu, Cr, Zr, Zn, Ta, Ti, Mg, Ni, Ca, Ba, Al, Y, Hf, Ce, Ti, Mo, W, Si, Pd, Ir, Sn, Fe, Pb, Pd, and Nd.

### Note 18:

The metal particles may include, in an amount of 5% by volume or less, particles of which the particles diameters are in the range of 2.0 μm to 0.005 μm, inclusive.

### Note 19:

The resin may be epoxy resin.

### Note 20:

Thermosetting resin may be used as a curing agent for the epoxy resin.

### Note 21:

The metal particles may be realized with one selected from between copper and tungsten.

According to at least one aspect of the embodiments described above, it is possible to produce the resin composition that has a low attenuation and makes the coating procedure possible.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A resin composition being a precursor of an acoustic matching layer for an ultrasound transducer element included in an ultrasound transducer element unit having an array transducer element including a piezoelectric body and an electrode, wherein
the resin composition includes resin and inorganic material particles, while satisfying
2.3≤Z/√ρ≤3.4
where Z denotes an acoustic impedance of the acoustic matching layer, whereas ρ denotes a density of the inorganic material particles.

2. The resin composition according to claim 1, wherein the inorganic material particles are ceramics particles.

3. The resin composition according to claim 1, wherein Z is in a range of 3.0 MRayl to 15 MRayl, inclusive.

4. The resin composition according to claim 2, wherein a particle diameter distribution of an average particle diameter of the ceramics particles is a unimodal distribution.

5. The resin composition according to claim 2, wherein a content amount of the ceramics particles in the resin composition is in a range of 20% by volume to 40% by volume, inclusive.

6. The resin composition according to claim 2, wherein an average particle diameter of the ceramics particles is in a range of 0.3 μm to 2 μm, inclusive.

7. The resin composition according to claim 2, wherein the ceramics particles contain a substance comprising at least one selected from the group consisting of Mg, Ca, Ba, B, Al, Y, Hf, Ce, Ti, W, and Si and at least one selected from the group consisting of O, C, N, and S.

8. The resin composition according to claim 2, wherein the ceramics particles include, in an amount of 5% by volume or less, particles of which particles diameters are in a range of 2.0 μm to 0.005 μm, inclusive.

9. The resin composition according to claim 1, wherein the resin is epoxy resin.

10. The resin composition according to claim 9, wherein thermosetting resin is used as a curing agent for the epoxy resin.

11. The resin composition according to claim 2, wherein the ceramics particles are realized with oxide aluminum.

12. The resin composition according to claim 2, wherein the ceramics particles are realized with tungsten carbide.

13. The resin composition according to claim 1, wherein
the inorganic material particles are metal particles satisfying
2.3≤Z/√ρ≤2.9
where Z denotes an acoustic impedance of the acoustic matching layer, whereas ρ denotes a density of the metal particles.

14. The resin composition according to claim 13, wherein a particle diameter distribution of an average particle diameter of the metal particles is a unimodal distribution.

15. The resin composition according to claim 13, wherein a content amount of the metal particles in the resin composition is in a range of 20% by volume to 40% by volume, inclusive.

16. The resin composition according to claim 13, wherein an average particle diameter of the metal particles is in a range of 1.0 μm to 4 μm, inclusive.

17. The resin composition according to claim 13, wherein the metal particles contain a substance comprising at least one selected from the group consisting of Au, Ag, Pt, Cu, Cr, Zr, Zn, Ta, Ti, Mg, Ni, Ca, Ba, Al, Y, Hf, Ce, Mo, W, Si, Pd, Ir, Sn, Fe, Pb, Pd, and Nd.

18. The resin composition according to claim 13, wherein the metal particles include, in an amount of 5% by volume or less, particles of which particles diameters are in a range of 2.0 μm to 0.005 μm, inclusive.

19. The resin composition according to claim 13, wherein the metal particles are realized with one selected from between copper and tungsten.
